(19) United States
(12) Patent Application Publication
(10) Pub. No.: US 2025/0391530 A1
(43) Pub. Date: Dec. 25, 2025

(54) A METHOD AND SYSTEM OF MECHANICAL VENTILATION THERAPY DATA MANAGEMENT

(71) Applicant: Shanghai SVM Medical Technology Co., Ltd., Shanghai (CN)

(72) Inventors: Yun LONG, Shanghai (CN); Xiaobo HUANG, Shanghai (CN); Bin DU, Shanghai (CN); Longxiang SU, Shanghai (CN); Yunping LAN, Shanghai (CN); Yingchuan LI, Shanghai (CN); You SHANG, Shanghai (CN); Chun PAN, Shanghai (CN); Hongli HE, Shanghai (CN); Qian ZHAI, Shanghai (CN); Jicheng ZHANG, Shanghai (CN); Huaiwu HE, Shanghai (CN); Yi CHI, Shanghai (CN); Man HUANG, Shanghai (CN); Xiaotong HAN, Shanghai (CN); Huanzhang SHAO, Shanghai (CN); Yugang ZHUANG, Shanghai (CN); Xiuling SHANG, Shanghai (CN); Song ZHANG, Shanghai (CN); Xiantao LI, Shanghai (CN); Ning SHEN, Shanghai (CN); Xiaoning HAO, Shanghai (CN); Zujun TANG, Shanghai (CN); Xianlong LIU, Shanghai (CN); Fuhong CAI, Shanghai (CN); Bin ZHU, Shanghai (CN)

(73) Assignee: Shanghai SVM Medical Technology Co., Ltd., Shanghai (CN)

(21) Appl. No.: 18/879,785

(22) PCT Filed: Nov. 23, 2022
(86) PCT No.: PCT/CN2022/133811

§ 371 Date: Dec. 29, 2024

(30) Foreign Application Priority Data
Jul. 1, 2022 (CN) 202210769891.1

(51) Int. Cl. G16H 15/00; G16H 10/60; G16H 20/40; G16H 40/20; G16H 50/20

(52) U.S. Cl. CPC G16H 15/00; G16H 10/60; G16H 20/40; G16H 40/20; G16H 50/20

(57) ABSTRACT

The present application relates to a method and system of mechanical ventilation therapy data management. The method includes: putting patient information and real-time received mechanical ventilation therapy data into a standard data structure object respectively to form standardized data; the mechanical ventilation therapy data comprises ventilator data; performing data cleaning on the standardized data to obtain a medical data set; performing data labeling on the mechanical ventilation therapy data in the medical data set; the data labeling comprises: marking amplitudes, frequencies, and shapes of waveform data, marking measurement values of numerical data, and marking abnormal events; when a report generation instruction is received, generating a mechanical ventilation therapy data analysis report based on the medical data set with the data labeling.

### CROSS-REFERENCE TO RELATED APPLICATIONS

This is a national stage of International Patent Application No. PCT/CN2022/133811, filed on Nov. 23, 2022, which claims priority to Chinese Patent Application No. 202210769891.1, filed on Jul. 1, 2022. The disclosures of the above-referenced applications are hereby incorporated by reference in their entirety.

### TECHNICAL FIELD

The present application relates to the field of mechanical ventilation therapy data management and real-time analysis technologies, and particularly relates to a method and system of mechanical ventilation therapy data management.

### BACKGROUND

Mechanical ventilation therapy technology has been widely used in intensive care units (ICU, NICU, RICU, EICU, etc.), anesthesiology departments and other departments in hospitals all over the world. Every year, tens of millions of critically ill patients around the world use this treatment technology, if used improperly, it will cause complications such as lung injury, ventilator-associated pneumonia, arrhythmia, acute heart failure, etc., aggravate the original disease, and endanger the patients' lives.

In the context of a common mechanical ventilation therapy, the patient is connected with a ventilator and a multi-parameter monitor, doctors and nurses observe closely and analyze the continuous mechanical ventilation waveform and numerical data on the ventilator screen to determine the working status of the ventilator, to analyze the changes in the patient's respiratory mechanics, the disease evolution, and the state of patient-ventilator interactions, and at the same time to observe the changes in physiological parameters of heart rate, blood pressure, blood oxygen saturation, and body temperature. It is also necessary to observe the blood gas analysis, laboratory diagnosis, imaging and other data through the clinical information systems (HIS/CIS/EMR system) in order to fully understand the disease status and evolution trend, adjust ventilator parameters and treatment strategies, prevent and control various complications.

However, each ventilator generates up to hundreds of MBs (Mega Bytes) of waveform data in 24 hours, doctors and nurses frequently check back and forth on the screens of various devices every day to observe and analyze all kinds of data, and at the same time, they also need to understand the patient's medical condition in the past time every day, and retrieve the 24/48-hours mechanical ventilation data, physiological parameters and other data during treatment from the devices or the clinical information systems. However, the devices and the clinical information systems only store numerical data, lack important waveform data, and contain a large amount of interference data, as well as data that the device itself analyzes incorrectly, so that doctors and nurses need to spend a lot of time reading and analyzing the data segment by segment, and performing identification and judgment on the data manually. Obviously, this clinical application way of the data causes heavy work pressure and requires a large amount of manpower, however, ventilators from various manufacturers are unable to provide 24-hours mechanical ventilation data analysis reports, while there is a general shortage of doctors and nurses in ICUs in hospitals around the world. So far, this situation has not changed.

In addition, one hospitals are often equipped with dozens to hundreds of ventilators and multi-parameter monitors from different manufacturers, there are complex problems such as different data communication protocols and data formats for similar devices, different data communication protocols and data formats for different kinds of devices, and clinical information systems are also from different manufacturers, which increase the difficulty of mechanical ventilation therapy data processing. Over the past decades, ventilator technology has developed continuously, but the clinical application way of the mechanical ventilation therapy data has not progressed, and there are significant shortcomings.

### SUMMARY

In view of the above analysis, embodiments of the present application aim at providing a method and system of mechanical ventilation therapy data management, to solve the problems of many difficulties caused by huge heterogeneous data to clinical application, poor medical quality of mechanical ventilation therapy, and low working efficiency of data processing by doctors and nurses.

In one aspect, the present application discloses a method of mechanical ventilation therapy data management, the method comprises:
putting patient information and real-time received mechanical ventilation therapy data into a standard data structure object respectively to form standardized data; the mechanical ventilation therapy data comprises ventilator data;
performing data cleaning on the standardized data to obtain a medical data set;
performing data labeling on the mechanical ventilation therapy data in the medical data set; the data labeling comprises: marking amplitudes, frequencies, and shapes of waveform data, marking measurement values of numerical data, and marking abnormal events;
when a report generation instruction is received, generating a mechanical ventilation therapy data analysis report based on the medical data set with the data labeling.

On the basis of the above solution, the present application also makes the following improvements:
further, the mechanical ventilation therapy data further comprises one or more of the following data: physiological parameters data, blood gas analysis data, laboratory diagnostic data, imaging data, clinical diagnosis and treatment information, and the ID (IDentity) of the device for outputting the mechanical ventilation therapy data; wherein
the physiological parameters data is output by multi-parameter monitors;
the patient information, the blood gas analysis data, the laboratory diagnostic data, the imaging data, and the clinical diagnosis and treatment information are all output by a clinical information system;
the mechanical ventilation data is output by ventilator devices.

Further, in the mechanical ventilation data, the waveform data comprises: mechanical ventilation pressure waveform data, volume waveform data, flow waveform data and end-tidal carbon dioxide waveform data; the numerical data comprises: airway resistance, work of breathing, compliance, ventilation parameters and respiratory rate;
in the data labeling process for the mechanical ventilation data,
the shapes of the labeled waveform data comprise: beginning of inspiratory phase, inspiratory phase, end of inspiratory phase, beginning of expiratory phase, expiratory phase, end of expiratory phase, peak airway pressure, and peak expiratory flow rate of each mechanical ventilation waveform, as well as inspiratory baseline, expiratory plateau height, and beginning and end of expiratory plateau of the end-tidal carbon dioxide waveform;
the labeled abnormal events comprise: patient-ventilator asynchronous event, high/low airway pressure, high/low positive end-expiratory pressure, high/low expired tidal volume, high/low minute ventilation, high/low inspired oxygen concentration, high/low oxygen concentration, high/low respiratory rate, high/low partial pressure of end-tidal carbon dioxide, high/low tidal volume, ventilators stop working, asphyxia and increasing airway pressure.

Further, in the physiological parameters data, the waveform data comprises:
photoplethysmogram waveform data and invasive arterial pressure waveform data;
the numerical data comprises: heart rate, blood pressure, blood oxygen saturation and body temperature;
in the data labeling process for the physiological parameters data, the shapes of the labeled electrocardiogram waveform data comprise: P wave, QRS complex, T wave, ST segment, PR interval, RR interval and QT interval of each ECG cycle waveform;
the shapes of the labeled respiratory waveform data comprise: peaks and valleys of each respiratory cycle waveform;
the shapes of the labeled photoplethysmogram waveform data comprise: peaks and valleys of each pulse cycle waveform;
the shapes of the labeled invasive arterial pressure waveform data comprise: peaks and valleys of each arterial systolic-diastolic cycle waveform;
the labeled abnormal events comprise: tachycardia, bradycardia, arrhythmia, cardiac arrest, atrial flutter, atrial fibrillation, ventricular flutter, ventricular fibrillation, atrioventricular block, ST-segment elevation/depression, high/low heart rate, high/low blood pressure, high/low body temperature and low blood oxygen saturation.

Further, in the blood gas analysis data, the numerical data comprises: partial pressure of oxygen and carbon dioxide, total carbon dioxide, potassium ion concentration, sodium ion concentration, bicarbonate ion concentration and pH value;
in the data labeling process for the blood gas analysis data,
the labeled abnormal events comprise: high/low partial pressure of oxygen, high/low partial pressure of carbon dioxide, high/low total carbon dioxide, high/low potassium ion concentration, high/low sodium ion concentration and high/low pH value.

Further, in the laboratory diagnostic data, the numerical data comprises: white blood cell count, red blood cell count, platelet count, hemoglobin, hematocrit, blood glucose, prothrombin time and activated partial thrombin time;
in the data labeling process for the laboratory diagnostic data,
the labeled abnormal events comprise: high/low white blood cell count, high/low red blood cell count, high/low hemoglobin, high/low hematocrit, high/low blood glucose, high/low platelet count, long/short prothrombin time, and long/short activated partial thrombin time.

Further, the reporting framework of the mechanical ventilation therapy data analysis report comprises a report overview page and a report analysis page; wherein, the report overview page is divided into a basic information area, a mechanical ventilation data area, and an analysis conclusion area;
the basic information area comprises at least the patient information and key time point information; and further comprises the following options: the clinical diagnosis information, the ID of the device for outputting the mechanical ventilation therapy data as well as details regarding mechanical ventilation mode and settings;
the mechanical ventilation data area comprises at least respiratory analysis results, patient-ventilator asynchronous events analysis results, airway resistance change analysis results, compliance change analysis results, and lung injury early warning analysis results; each of the analysis results of the mechanical ventilation data area is obtained based on the mechanical ventilation data and its data labeling; the respiratory analysis results comprise a rapid shallow breathing index;
the analysis conclusion area is configured to display the analysis descriptions and conclusions of the mechanical ventilation therapy data;
the report analysis page is configured to display a detailed analysis report of one or more mechanical ventilation therapy data separately in graphical form.

Further, the report analysis page comprises a trend graph page; the trend graph page comprises:
trend analysis areas of pressure, volume, and ventilation volume of the mechanical ventilation data;
a trend analysis area of a respiratory frequency;
a trend analysis area of an inhaled oxygen concentration; and,
a trend analysis description and conclusion area, configured to synthesize the trend direction of each trend analysis area to provide trend analysis descriptions and the corresponding conclusions;
the content of each trend analysis area in the trend graph page is obtained based on the mechanical ventilation data and its data labeling.

Further, the report analysis page further comprises a data page;
the data page comprises at least:
a mechanical ventilation setting parameters data sub-page, comprising: a mechanical ventilation setting parameters record sheet, ventilation modes of mechanical ventilation and a change trend graph of setting parameters;
a mechanical ventilation monitoring parameters data sub-page, comprising a mechanical ventilation monitoring parameters record sheet;
the contents of the mechanical ventilation setting parameters data sub-page and the mechanical ventilation monitoring parameters data sub-page are obtained based on the mechanical ventilation data and its data labeling;
the data page further comprises the following optional data sub-pages:
a 24-hour physiological parameters data sub-page, comprising a 24-hour vital signs monitoring record sheet and a heart rate/respiratory rate/blood pressure trend graph; the contents of the 24-hour physiological parameters data sub-page are obtained based on the physiological parameters data and its data labeling;
a 24-hour blood gas analysis and laboratory diagnostic data sub-page, comprising a 24-hour blood gas analysis monitoring record sheet, a partial pressure of oxygen/partial pressure of carbon dioxide/bicarbonate trend graph, and a laboratory diagnostic data sheet;
the contents of the 24-hour blood gas analysis and laboratory diagnostic data sub-page are obtained based on the blood gas analysis data, the laboratory diagnostic data, and their data labeling;
an alarm events data sub-page, comprising: an alarm events record sheet, a 24-hour alarm events distribution diagram, and an alarm events comprehensive analysis sheet;
the alarm events data sub-page is obtained based on the abnormal events labeled by the mechanical ventilation therapy data;
an extended image data sub-page, configured to display the image data.

Further, the report analysis page further comprises a graphic page; the graphic page is divided into a mechanical ventilation waveform data area, a mechanical ventilation loop chart area, and a physiological parameters waveform data area; wherein
the mechanical ventilation waveform data area comprises waveform graphs of continuous normal mechanical ventilation data and waveform graphs of abnormal mechanical ventilation events data;
the mechanical ventilation loop chart area comprises a pressure-volume loop chart and a flow-volume loop chart drawn by the combination of the mechanical ventilation pressure, volume, and flow data;
the contents of the mechanical ventilation waveform data area and the mechanical ventilation loop chart area are obtained based on the mechanical ventilation data and its data labeling;
the physiological parameters waveform data area comprises waveform graphs of continuous physiological parameters data synchronized with the mechanical ventilation data waveform graphs;
the contents of the physiological parameters waveform data area are obtained based on the physiological parameters data and its data labeling.

Further, the report generation instruction is a customized report generation instruction;
the customized report generation instruction comprises one or more customized parameters, configured to generate the report overview page and the report analysis page matching the customized parameters;
the customized parameters comprise the trend graph page, the data page, or the graphic page.

Further, the method further comprises:
in the data labeling process, a first aid instruction is issued when one or more abnormal events of ventilators stop working, asphyxia, increasing airway pressure, cardiac arrest, tachycardia, bradycardia, ventricular flutter, ventricular fibrillation and hypotension occur, the first aid instruction carries the information about the abnormal events that occur.

Further, the method further comprises:
in the data labeling process, a ventilator parameters adjustment instruction is issued when one or more abnormal events of patient-ventilator asynchronous event, high airway pressure, low airway pressure, high positive end expiratory pressure, low positive end expiratory pressure, high minute ventilation, low minute ventilation, high tidal volume, low tidal volume, high respiratory rate, low respiratory rate, high oxygen concentration and low oxygen concentration occur, the ventilator parameters adjustment instruction carries the information about the abnormal events that occur.

Further, in the standard data structure object, a corresponding data structure object is configured for each item of the mechanical ventilation therapy data and the patient information respectively;
each decoupled mechanical ventilation therapy data and the patient information are placed into the corresponding data structure object to form standardized data.

Further, while receiving the mechanical ventilation therapy data in real time, streaming media data of the treatment scenes is also received in real time;
live broadcast in real time of the waveform data, the numeric data, and the treatment scenes in the mechanical ventilation therapy data of the medical data set as well as the mechanical ventilation therapy data analysis reports of some or all patients in one or more wards.

In another aspect, the present application also provides a system of mechanical ventilation therapy data management, the system comprises an application software system, clinical data terminal devices and a database server; wherein,
the application software system comprises:
a data acquisition and standardization unit, configured to put the patient information and the real-time received mechanical ventilation therapy data into a standard data structure object respectively to form standardized data; the mechanical ventilation therapy data comprises the mechanical ventilation data;
a data cleaning unit, configured to perform data cleaning on the standardized data to obtain a medical data set;
a data labeling unit, configured to perform data labeling on the mechanical ventilation therapy data in the medical data set;
the data labeling comprises: marking amplitudes, frequencies, and shapes of waveform data, marking measurement values of numerical data, and marking abnormal events;
a report generation unit, configured to generate a mechanical ventilation therapy data analysis report based on the medical data set with the data labeling when a report generation instruction is received;
the clinical data terminal devices, configured to issue the report generation instructions, and further configured to receive and display the mechanical ventilation therapy data analysis reports;
the database server, configured to store the standardized data, the medical data sets, and the mechanical ventilation therapy data analysis reports in partitions.

On the basis of the above solution, the present application also makes the following improvements.

Further, the mechanical ventilation therapy data further comprises one or more of the following: physiological parameters data, blood gas analysis data, laboratory diagnostic data, imaging data, clinical diagnosis and treatment information, and the ID of the device for outputting the mechanical ventilation therapy data; wherein,
the physiological parameters data is output by multi-parameter monitors;
the patient information, the blood gas analysis data, the laboratory diagnostic data, the imaging data and the clinical diagnosis and treatment information are all output by a clinical information system;
the mechanical ventilation data is output by ventilator devices.

Further, in the mechanical ventilation data, the waveform data comprises: mechanical ventilation pressure waveform data, volume waveform data, flow waveform data and end-tidal carbon dioxide waveform data; the numerical data comprises: airway resistance, work of breathing, compliance, ventilation parameters and respiratory rate;
in the data labeling process for the mechanical ventilation data,
the shapes of the labeled waveform data comprise: beginning of inspiratory phase, inspiratory phase, end of inspiratory phase, beginning of expiratory phase, expiratory phase, end of expiratory phase, peak airway pressure, and peak expiratory flow rate of each mechanical ventilation waveform, as well as inspiratory baseline, expiratory plateau height, and expiratory plateau start and end of the end-tidal carbon dioxide waveform;
the labeled abnormal events comprise: patient-ventilator asynchronous event, high/low airway pressure, high/low positive end expiratory pressure, high/low expired tidal volume, high/low minute ventilation, high/low inspired oxygen concentration, high/low oxygen concentration, high/low respiratory rate, high/low partial pressure of end-tidal carbon dioxide, high/low tidal volume, ventilators stop working, asphyxia and increasing airway pressure.

Further, in the physiological parameters data, the waveform data comprises: electrocardiogram waveform data, respiratory waveform data, photoplethysmogram waveform data and invasive arterial pressure waveform data; the numerical data comprises: heart rate, blood pressure, blood oxygen saturation and body temperature;
in the data labeling process for the physiological parameters data,
the shapes of the labeled electrocardiogram waveform data comprise: P wave, QRS complex, T wave, ST segment, PR interval, RR interval and QT interval of each ECG cycle waveform;
the shapes of the labeled respiratory waveform data comprise: peaks and valleys of each respiratory cycle waveform;
the shapes of the labeled photoplethysmogram waveform data comprise: peaks and valleys of each pulse cycle waveform;
the shapes of the labeled invasive arterial pressure waveform data comprise: peaks and valleys of each arterial systolic-diastolic cycle waveform;
the labeled abnormal events comprise: tachycardia, bradycardia, arrhythmia, cardiac arrest, atrial flutter, atrial fibrillation, ventricular flutter, ventricular fibrillation, atrioventricular block, ST-segment elevation/depression, high/low heart rate, high/low blood pressure, high/low body temperature and low blood oxygen saturation.

Further, in the blood gas analysis data, the numerical data comprises: partial pressure of oxygen and carbon dioxide, total carbon dioxide, potassium ion concentration, sodium ion concentration, bicarbonate ion concentration and pH value;
in the data labeling process for the blood gas analysis data,
the labeled abnormal events comprise: high/low partial pressure of oxygen, high/low partial pressure of carbon dioxide, high/low total carbon dioxide, high/low potassium ion concentration, high/low sodium ion concentration and high/low pH value.

Further, in the laboratory diagnostic data, the numerical data comprises: white blood cell count, red blood cell count, platelet count, hemoglobin, hematocrit, blood glucose, prothrombin time and activated partial thrombin time;
in the data labeling process for the laboratory diagnostic data,
the labeled abnormal events comprise: high/low white blood cell count, high/low red blood cell count, high/low hemoglobin, high/low hematocrit, high/low blood glucose, high/low platelet count, long/short prothrombin time, and long/short activated partial thrombin time.

Further, the reporting framework of the mechanical ventilation therapy data analysis report comprises a report overview page and a report analysis page; wherein, the report overview page is divided into a basic information area, a mechanical ventilation data area, and an analysis conclusion area;
the basic information area comprises at least the patient information and key time point information; and further comprises the following options: the clinical diagnosis information, the ID of the device for the mechanical ventilation therapy data as well as mechanical ventilation mode and setting information;
the mechanical ventilation data area comprises at least respiratory analysis results, patient-ventilator asynchronous events analysis results, airway resistance change analysis results, compliance change analysis results, and lung injury early warning analysis results; each of the analysis results of the mechanical ventilation data area is obtained based on the mechanical ventilation data and its data labeling; the respiratory analysis results comprise a rapid shallow breathing index;
the analysis conclusion area is configured to display the analysis descriptions and conclusions of the mechanical ventilation therapy data;
the report analysis page is configured to display a detailed analysis report of one or more mechanical ventilation therapy data separately in graphical form.

Further, the report analysis page comprises a trend graph page; the trend graph page comprises:
trend analysis areas of pressure, volume, and ventilation volume of the mechanical ventilation data;
a trend analysis area of a respiratory frequency;
a trend analysis area of an inhaled oxygen concentration; and,
a trend analysis description and conclusion area, configured to synthesize the trend direction of each trend analysis area to provide trend analysis descriptions and the corresponding conclusions;
the content of each trend analysis area in the trend graph page is obtained based on the mechanical ventilation data and its data labeling.

Further, the report analysis page further comprises a data page;
the data page comprises at least:
a mechanical ventilation setting parameters data sub-page, comprising: a mechanical ventilation setting parameters record sheet, ventilation modes of mechanical ventilation and a change trend graph for setting parameters;
a mechanical ventilation monitoring parameters data sub-page, comprising a mechanical ventilation monitoring parameters record sheet;
the contents of the mechanical ventilation setting parameters data sub-page and the mechanical ventilation monitoring parameters data sub-page are obtained based on the mechanical ventilation data and its data labeling;
the data page further comprises the following optional data sub-pages:
a 24-hour physiological parameters data sub-page, comprising a 24-hour vital signs monitoring record sheet and a heart rate/respiratory rate/blood pressure trend graph; the contents of the 24-hour physiological parameters data sub-page are obtained based on the physiological parameters data and its data labeling;
a 24-hour blood gas analysis and laboratory diagnostic data sub-page, comprising a 24-hour blood gas analysis monitoring record sheet, a partial pressure of oxygen/partial pressure of carbon dioxide/bicarbonate trend graphs, and a laboratory diagnostic data sheet;
the contents of the 24-hour blood gas analysis and laboratory diagnostic data sub-page are obtained based on the blood gas analysis data, the laboratory diagnostic data, and their data labeling;
an alarm events data sub-page, comprising: an alarm events record sheet, 24-hour alarm events distribution diagram, and an alarm events comprehensive analysis sheet;
the alarm events data sub-page is obtained based on the abnormal events labeled by the mechanical ventilation therapy data;
an extended image data sub-page, configured to display the image data.

Further, the report analysis page further comprises a graphic page; the graphic page is divided into a mechanical ventilation waveform data area, a mechanical ventilation loop chart area, and a physiological parameters waveform data area; wherein,
the mechanical ventilation waveform data area comprises waveform graphs of continuous normal mechanical ventilation data and waveform graphs of abnormal mechanical ventilation events data;
the mechanical ventilation loop chart area comprises a pressure-volume loop chart and a flow-volume loop chart drawn by the combination of the mechanical ventilation pressure, volume, and flow data;
the contents of the mechanical ventilation waveform data area and the mechanical ventilation loop chart area are obtained based on the mechanical ventilation data and its data labeling;
the physiological parameters waveform data area comprises waveform graphs of continuous physiological parameters data synchronized with the mechanical ventilation data waveform graphs;
the content of the physiological parameters waveform data area is obtained based on the physiological parameters data and its data labeling.

Further, the report generation instruction is a customized report generation instruction;
the customized report generation instruction comprises one or more customized parameters, configured to generate the report overview page and the report analysis page matching the customized parameters;
the customized parameters comprise the trend graph page, the data page, or the graphic page.

Further, in the data labeling process of the data labeling unit, a first aid instruction is issued to the clinical data terminal devices when one or more abnormal events of ventilators stop working, asphyxia, increasing airway pressure, cardiac arrest, tachycardia, bradycardia, ventricular flutter, ventricular fibrillation and hypotension occur, the first aid instruction carries the information about the abnormal events that occur;
the clinical data terminal devices are further configured to receive and respond to the first aid instructions.

Further, in the data labeling process of the data labeling unit, a ventilator parameters adjustment instruction is issued to the clinical data terminal devices when one or more abnormal events of patient-ventilator asynchronous event, high airway pressure, low airway pressure, high positive end expiratory pressure, low positive end expiratory pressure, high minute ventilation, low minute ventilation, high tidal volume, low tidal volume, high respiratory rate, low respiratory rate, high oxygen concentration and low oxygen concentration occur, the ventilator parameter adjustment instruction carries the information about the abnormal events that occur;
the clinical data terminal devices are further configured to receive and respond to the ventilator parameters adjustment instructions.

Further, in the standard data structure object, a corresponding data structure object is configured for each item of the mechanical ventilation therapy data and the patient information respectively;
each decoupled mechanical ventilation therapy data and the patient information are placed into the corresponding data structure object to form standardized data.

Further, the application software system further comprises a streaming media management unit, the streaming media management unit is configured to receive streaming media data of the treatment scenes in real time; the streaming media data is output by bedside audio video devices;
the database server, further configured to store the streaming media data in partitions and associate the streaming media data with the medical data sets and the mechanical ventilation therapy data analysis reports;
the clinical data terminal devices are further configured to live broadcast in real time of the waveform data, the numeric data, and the treatment scenes in the mechanical ventilation therapy data of the medical data set as well as the mechanical ventilation therapy data analysis reports of some or all patients in one or more wards.

Further, the system further comprises a multi-mode gateway and a master control server; wherein,
the multi-mode gateway is configured to connect the ventilator devices, the multi-parameter monitors, the bedside audio video devices, the clinical information systems and the clinical data terminal devices from different manufacturers in each ward of the hospital through the network; the multi-mode gateway is embedded with a variety of communication protocols to realize the data communication between the ventilator devices, the multi-parameter monitors, the bedside audio video devices, the clinical information systems and the data acquisition and standardization unit, and configured to realize the data communication between the application software system and the clinical data terminal devices where the patients are located;
the master control server, embedded with a multi-task operating system and an application software system running on the multi-task operating system, configured to control the operation of the application software system.

Compared with existing technology, the present application achieves at least one of the following beneficial effects.

The method and system of mechanical ventilation therapy data management provided by the present application has the following advantages.

1. The present application provides technical means for doctors and nurses to centrally manage the mechanical ventilation therapy data, doctors and nurses use clinical data terminal devices to remotely and centrally manage the mechanical ventilation therapy data business of patients in a plurality of wards in a visualized way, providing real-time data monitoring and mechanical ventilation data analysis reports, which improves data work efficiency and mechanical ventilation therapy quality, reduces the labor intensity of doctors and nurses, alleviates the problem of human resources shortage in ICU, solves the defects of the existing mechanical ventilation therapy business, and has wide applicability.

2. The present application decouples the different data communication protocols from various manufacturers' devices from the contained data, separates the multi-source heterogeneous data, parses and filters the huge multi-source heterogeneous data separately, and reconstructs the data into standardized data, which effectively reduces the complexity of the parsing process, improves the parsing speed and stability, solves the defects that the existing communication protocols of the medical devices data and the data formats are confusing and difficult to be uniformly processed, and has wide applicability.

3. The present application aggregates and analyzes the mechanical ventilation therapy data to generate the mechanical ventilation therapy data analysis reports, so that doctors and nurses can conveniently obtain summarized data sets, can quickly and accurately judge the current and past mechanical ventilation therapy status and disease evolution trends, and provide timely intervention measures, solves the difficulties caused by huge multi-source heterogeneous data to the clinical application, reduces the work pressure of doctors and nurses, improves the quality and efficiency of medical treatment, and has wide applicability.

4. The data labeling method provided by the present application can effectively remove the interference data and the data that the device itself analyzes incorrectly, effectively reduce the pressure of the data work of doctors and nurses, and at the same time substantially improve the data quality, solve the defects that the existing devices and clinical information systems store the data of low quality, poor usability, and lack of waveform data, having wide applicability.

In the present invention, the above technical solutions can also be combined, to implement more preferred combined solutions. Other features and advantages of the present invention will be described in the subsequent specification, and part of the advantages can become apparent from the specification, or be understood through the implementation of the present invention. The objects and other advantages of the present invention can be implemented and obtained from the contents particularly illustrated in the specification and the drawings.

### BRIEF DESCRIPTION OF THE DRAWINGS

The drawings are merely for the purpose of illustrating the particular embodiments, and are not considered as limitation to the present application.

Throughout the drawings, the same reference signs denote the same elements.

FIG. 1 is a flow chart of the method of mechanical ventilation therapy data management provided in embodiment 1 of the present application.

FIG. 2 is an example diagram of the standard data structure object provided in embodiment 1 of the present application.

FIG. 3 is a schematic diagram of the model input processing during the process of determining the patient-ventilator asynchronous event provided in embodiment 1 of the present application; wherein FIG. 3(a) represents a schematic diagram of the two times sliding window processing; FIG. 3(b) represents a schematic diagram of the time series after the two times sliding window processing.

FIG. 4 is the report overview page of the mechanical ventilation therapy data analysis provided in embodiment 1 of the present application.

FIG. 5 is the trend graph page of the mechanical ventilation therapy data analysis report provided in embodiment 1 of the present application.

FIG. 6 is the data page of the mechanical ventilation therapy data analysis report provided in embodiment 1 of the present application; wherein, FIG. 6(a) represents a data sub-page of the mechanical ventilation setting parameters; FIG. 6(b) represents a data sub-page of the mechanical ventilation monitoring parameters;

FIG. 6(c) represents a data sub-page of the 24-hour physiological parameters; FIG. 6(d) represents data sub-page of the 24-hour blood gas analysis and laboratory diagnostic; FIG. 6(e) represents a data sub-page of the alarm events; FIG. 6(f) represents the image data sub-page.

FIG. 7 is the graphic page of the mechanical ventilation therapy data analysis report provided in embodiment 1 of the present application.

FIG. 8 is an architectural diagram of the system of mechanical ventilation therapy data management with data aggregation of multiple devices provided in embodiment 2 of the present application.

FIG. 9 is an implementation example of the patient displayed in a visualized way provided in embodiment 2 of the present application.

FIG. 10 is an implementation example of the ward management panel of the clinical data terminal devices provided in embodiment 2 of the present application.

FIG. 11 is an implementation example of the data analysis report panel of the clinical data terminal devices provided in embodiment 2 of the present application.

FIG. 12 is an architectural diagram of the system of mechanical ventilation therapy data management in the form of edge computing nodes provided in embodiment 3 of the present application.

### DETAILED DESCRIPTION

The preferable embodiments of the present application will be particularly described below by referring to the drawings. The drawings form part of the present application, are used to explain the principle of the present application together with the embodiments of the present application, and are not limiting the scope of the present application.

### Embodiment 1

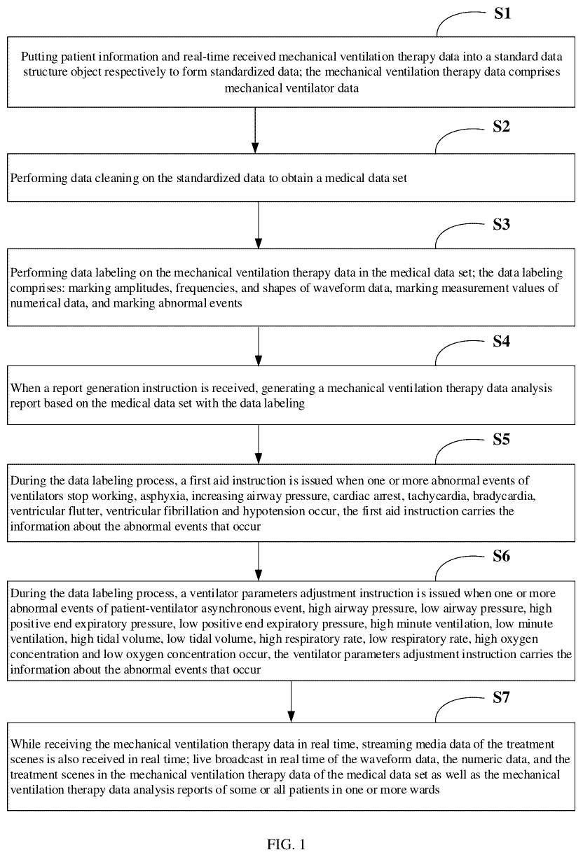

Embodiment 1 of the present application provides a method of mechanical ventilation therapy data management, the flow chart as shown in FIG. 1, comprises the following steps.

Step S1: putting patient information and real-time received mechanical ventilation therapy data into a standard data structure object respectively to form standardized data; the mechanical ventilation therapy data comprises ventilator data.

In step S1, the mechanical ventilation therapy data further comprises one or more of the following data: physiological parameters data, blood gas analysis data, laboratory diagnostic data, imaging data, clinical diagnosis and treatment information, and the ID of the device for outputting the mechanical ventilation therapy data; wherein the physiological parameters data is output by multi-parameter monitors; the patient information, the blood gas analysis data, the laboratory diagnostic data, the imaging data and the clinical diagnosis and treatment information are all output by a clinical information system; the mechanical ventilation data is output by ventilator devices. For example, the clinical information system comprises at least one of HIS, CIS, and EMR systems.

In the specific implementation process, in order to solve the problems that the output data forms of similar devices from different manufacturers as well as of different kinds of mechanical ventilation therapy data acquisition devices are not uniform, resulting in cumbersome subsequent data processing procedure, the present embodiment sets up a standard data structure object so as to perform standardize processing on the multi-source heterogeneous data output from the above devices. Specifically, in the present embodiment, different data communication protocols of the devices from various manufacturers are received and processed in parallel; data messages from the ventilators, the multi-parameter monitors and the clinical information systems are synchronously collected; the head and tail structures and data portions in the data messages are decoupled and separated, wherein the data portions are parsed in parallel, from which the valid data is extracted and filled into the corresponding positions of the preset standard data structure object to form standardized data, which is bundled and encapsulated with the patient information, setting the unique identification, and storing in the database; the standard data structure object is an abstraction of the mechanical ventilation therapy data and the patient information, simplifying the complex multi-source heterogeneous data, thereby improving the efficiency of the data analysis, processing, and utilization, reducing the cost of data application. Therefore, the present embodiment can complete the standardization process of the multi-source heterogeneous data by performing data parsing and standardization processing, reduce the complexity of parsing multi-source heterogeneous data, improve the speed and stability of data parsing, the standardized data can not only conserve storage space, but also improve the efficiency of data computing and retrieval.

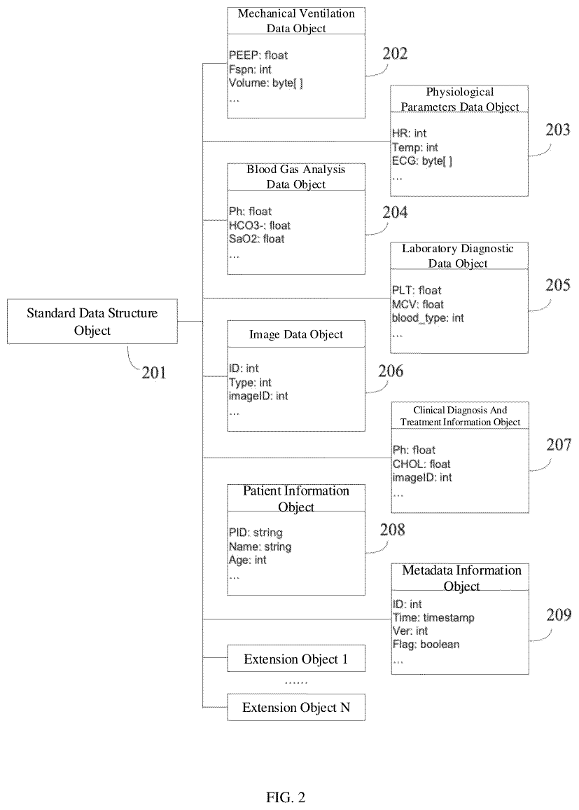

FIG. 2 illustrates an example of a standard data structure object 201, comprising a mechanical ventilation data object 202, a physiological parameters data object 203, a blood gas analysis data object 204, a laboratory diagnostic data object 205, an image data object 206, a clinical diagnosis and treatment information object 207 and a patient information object 208, the specific contents of each data structure object can be preset according to the actual situation. In addition, FIG. 2 further comprises a metadata information object 209, configured to describe the standard data structure object 201 itself and the contained structures, contents and attributes of the data structure object, and define the names, relationships, fields, constraints and syntactic structures of each data structure object. Since the blood gas analysis data, the laboratory diagnostic data, the image data, etc. are all periodic data that occur intermittently or at regular intervals during the mechanical ventilation therapy process, the standard data structure object 201 allows to be set to null when the relevant data does not occur. In addition, in order to enhance the adaptability and extensibility of the standard data structure object 201, the standard data structure object 201 comprises the extension objects 1-n as shown in FIG. 2, which are configured to add other relevant mechanical ventilation therapy data. For example, for a shock patient with mechanical ventilation therapy, filtration blood of CRRT and dynamic monitoring of urine volume need to be performed at the same time, at this time, the mechanical ventilation therapy data can also comprise continuous blood filtration data and dynamic monitoring data of urine volume, accordingly, the standard data structure object 201 can extend the continuous blood filtration data object and the dynamic monitoring data object of urine volume, so as to add other relevant mechanical ventilation therapy data conveniently. The standard data structure object provided by the present embodiment effectively solved the defects that the existing communication protocols of the medical devices data and the data formats are confusing and difficult to be uniformly processed, and have wide applicability. Specifically, the standard data structure object can be implemented by high-level languages, taking the object-oriented programming language JAVA as an example, the standard data structure object is a specific class, the attributes (fields) of the class are the various data structure objects, the various method functions of the class are programmed for external calls, configured to set the specific data structure objects and their contained parameters, for example, airway resistance and work of breathing represented by Float type, volume waveform data represented by LinkedList type, attribute identifiers represented by Boolean type. The standard data structure object class is distributed to the parsing programs, each parsing program creates a standard data structure class object for each data packet when parsing in parallel, calls the methods of the class to fill the data, and combines the standard data structure class objects at the same time into a single standardized data through the aggregation program.

Step S2: performing data cleaning on the standardized data to obtain a medical data set.

Data cleansing is a process for re-examining and verification data, aiming to delete duplicate information, correct existing errors, and provide data consistency. The existing data cleaning techniques are relatively mature, in the actual implementation process, a corresponding data cleaning method can be selected according to the specific application scene and the specific situation of the mechanical ventilation therapy data under the application scene. For example, the data cleaning process in the present embodiment can comprise the following operations:
(1) judgment of data constraint range: range constraints on medical theory data values, data constraints on empiricist extrapolations;
(2) conversion of data unit/data precision;
(3) removal of data outlier;
(4) for waveform data, also performing data length calibration, data integrity verification, etc.;
the medical data set obtained after data cleaning can be used for subsequent data analysis.

Step S3: performing data labeling on waveform data and numerical data of the mechanical ventilation therapy data; the data labeling comprises: marking amplitudes, frequencies, and shapes of waveform data, marking measurement values of numerical data, and marking abnormal events; wherein the measurement values of the labeled numerical data can comprise parameters that are higher than the upper limit threshold of the corresponding numerical data or lower than the lower limit threshold of the corresponding numerical data; the labeled data is automatically stored into the medical data set.

Since the mechanical ventilation therapy data can contain many specific data that cannot be exhaustively listed, in the present embodiment, only common data and common data labeling in various mechanical ventilation therapy data are highlighted.

Specifically, (1) in the mechanical ventilation data, the waveform data comprises: mechanical ventilation pressure waveform data, volume waveform data, flow waveform data and end-tidal carbon dioxide waveform data; the numerical data comprises: airway resistance, work of breathing, compliance, ventilation parameters and respiratory rate; in the data labeling process for the mechanical ventilation data, the shapes of the labeled waveform data comprises: beginning of inspiratory phase, inspiratory phase, end of inspiratory phase, beginning of expiratory phase, expiratory phase, end of expiratory phase, peak airway pressure, and peak expiratory flow rate of each mechanical ventilation waveform, as well as inspiratory baseline, expiratory plateau height, and expiratory plateau beginning and end of the end-tidal carbon dioxide waveform; the labeled contents can be configured to generate the report overview page and the report analysis page either directly or after statistics, computation and analysis; for example, the peak airway pressure and the peak expiratory flow rate can be determined by the following process.

(a) Firstly, common filtering algorithms such as median/homogenous filtering are adopted to filter and aggregate the mechanical ventilation pressure, volume and flow waveform data having high sampling rate in order to reduce the complexity of waveform processing. A pressure-time curve of the mechanical ventilation pressure waveform data is recorded as P(t), a volume-time curve of the mechanical ventilation volume waveform data is recorded as V(t), a flow-time curve of the mechanical ventilation flow waveform data is recorded as F(t) setting the observation window, for example, it takes at least 3.428s for two breaths, so the observation window duration is longer than 3.428s, for example, the observation window can be set to 4s. The following steps (b)-(d) are all calculated according to the observation window.

(b) In the i-th observation window, performing the following process to the volume-time curve V(t).
finding the peaks and valleys within the observation window using the peak finding algorithm and storing the results, and obtaining:
the i-th inspiratory start time

$${t}_{i}^{ins} ,$$

the i-th inspiratory end time

$${t}_{i}^{exp} ,$$

wherein

$${t}_{i}^{exp}$$

is also the i-th expiratory start time;
the i-th expiratory end time

$${t}_{i + 1}^{i n s} ,$$

wherein

$${t}_{i + 1}^{ins}$$

is also the i+1-th inspiratory start time.

(c) According to the above start and end time of the inspiratory phase and the expiratory phase, performing the calculation as follows to the pressure-time curve P(t) to obtain the peak airway pressure PIP, and storing the results; the i-th peak airway pressure:

$${PIP}_{i} = Max ( P ( t ) \vert t ∈ ( {t}_{i}^{ins} , {t}_{i}^{exp} ) ) ;$$

t represents the time.

(d) According to the above start and end time of inspiratory phase and expiratory phase, performing the calculation as follows to the flow-time curve F(t) to obtain the peak expiratory flow rate PEF, and storing the results; the i-th peak expiratory flow rate is:

$${PEF}_{i} = Min ( F ( t ) \vert t ∈ ( {t}_{i}^{exp} , {t}_{i + 1}^{ins} ) ) .$$

The measurement values of the labeled numerical data comprise: parameters that are higher than the upper limit threshold of the corresponding numerical data or lower than the lower limit threshold of the corresponding numerical data; the labeled content can be configured to generate the trend graph page, sub-page of the mechanical ventilation setting parameters and sub-page of the mechanical ventilation monitoring parameters either directly or after statistics, computation and analysis; for example, numerical comparison is continuously performed between the compliance data and its corresponding upper and lower limit thresholds, when the compliance data is below the lower limit threshold (e.g., the lower limit threshold is 25 ml/cmH₂O), the compliance data and the occurrence time at that moment are labeled and stored.

The labeled abnormal events comprise: patient-ventilator asynchronous event, high/low airway pressure, high/low positive end-expiratory pressure, high/low expired tidal volume, high/low minute ventilation, high/low inspired oxygen concentration, high/low oxygen concentration, high/low respiratory rate, high/low partial pressure of end-tidal carbon dioxide, high/low tidal volume, ventilators stop working, asphyxia and increasing airway pressure. The labeled contents can be configured to generate the report overview page, the alarm events data sub-page, and the graphic page either directly or after statistics, computation and analysis.

For example, the patient-ventilator asynchronous event can be determined based on a neural network model, the specific process is described as below.

(a) Constructing the model inputs based on the pressure-time curve P(t), the volume-time curve V(t), and the flow-time curve F(t).

Specifically, the pressure-time curve P(t), the volume-time curve V(t) and the flow-time curve F(t) form a three-dimensional time series as the model inputs: T= [symbol] S₁, . . . , S_{c}, . . . , S_{C} [symbol] , wherein S_{c} represents the input vector of the c-th sampling point, C represents the total number of the sampling points processed by the neural network model in a single run, that is, the length of the model inputs;

$${S}_{c} = ( {S}_{c}^{1} , {S}_{c}^{2} , {S}_{c}^{3} ) , {S}_{c}^{1} , {S}_{c}^{2} , {S}_{c}^{3}$$

represents the values of flow rate, pressure, and volume at the c-th sampling point respectively.

(b) Performing two times sliding window processing for feature enrichment to obtain the time series after sliding window processing.

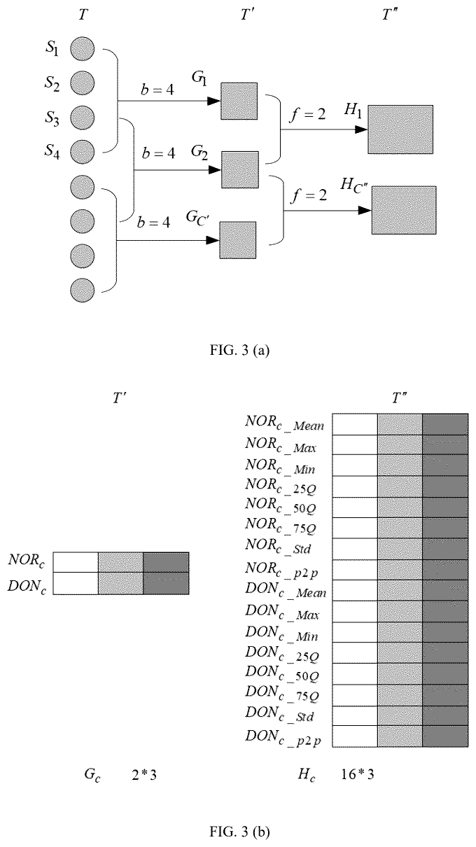

Two times sliding window processing: considering the intrinsic dependence of the time series data, the present embodiment adopts two times sliding window to process the sequence data in pieces, and extract the statistical features of each window, to exclude interfering factors and enhance the feature rules of the time series, thereby better describing the centralized tendency and discrete degree of the distribution of the data values of the waveform sampling points, the skewness and kurtosis features of the waveform shape changes, so that it is convenient to capture the dependency features and local features of the input sequence subsequently. The schematic diagram of the two times sliding window processing is shown in FIG. 3(a).

Wherein, the first sliding window processing, executing the following steps: performing the sliding window processing to the model input T with b/2 as the sliding window step, b represents the width of the first sliding window; after the first sliding window processing, the model input length is changed from C to C′, the values range of c is changed to [1, C′];

$${C}^{′} = \frac{2 * C - b}{b} ;$$

after the first sliding window processing, based on the class l waveform data in all sampling points within the c-th sliding window, the Euclidean distance

$${NOR}_{c}^{l}$$

of the class l waveform data in the Euclidean matrix vector is calculated, that is,

$${NOR}_{c}^{l} = \sqrt{{( {S}_{c}^{l} )}^{2} + {( {S}_{c + 1}^{l} )}^{2} + … + {( {S}_{c + b - 1}^{l} )}^{2}} ,$$

l takes 1, 2, 3 respectively;
finally, the Euclidean matrix vector

$${NOR}_{c} = ( {NOR}_{c}^{1} , {NOR}_{c}^{2} , {NOR}_{c}^{3} )$$

is obtained.

Based on the class l waveform data in all sampling points within the c-th sliding window, the distance difference

$${DON}_{c}^{l}$$

of the class l waveform data in the distance difference vector is calculated, that is,

$${DON}_{c}^{l} = {NOR}_{c + 1}^{l} - {NOR}_{c}^{l} ;$$

finally, the distance difference vector is obtained and represented as

$${DON}_{c} = ( {DON}_{c}^{1} , {DON}_{c}^{2} , {DON}_{c}^{3} ) ;$$

therefore, the time series after the first sliding window processing: T′= [symbol] G₁, . . . , G_{c}, . . . , G_{C′} [symbol] , wherein, G_{c}= [symbol] NOR_{c}, DON_{c} [symbol] .

The second sliding window processing, executing the following steps:
performing the sliding window processing to the time series T′ again with f/2 as the sliding window step, f represents the width of the second time sliding window; after the second time sliding window processing, the model input length is changed from C′ to C″, the values range of c is changed to [1,c″];

$${C}^{″} = \frac{2 * {C}^{′} - f}{f} ;$$

during the second sliding window processing, the derived features of all the Euclidean matrix vectors and distance difference vectors within the second time sliding window can be obtained, which can be illustrated by taking the c-th sliding window as an example, we can obtain:
the mean vector of the Euclidean matrix and the distance difference

$${NOR}_{c - Mean} , {DON}_{c - Mean} ) ,$$

the maximum vector of the Euclidean matrix and the distance difference

$$( N O {R}_{c - Max} , DO {N}_{c - Max} ) ,$$

the minimum vector of the Euclidean matrix and the distance difference

$$( NO {R}_{c - Min} , DO {N}_{c - Min} ) ,$$

the upper quartile vector of the Euclidean matrix and the distance difference

$$( {NOR}_{c - 25 Q} , {DON}_{c - 25 Q} ) ,$$

the median vector of the Euclidean matrix and the distance difference

$$( {NOR}_{c - 50 Q} , {DON}_{c - 50 Q} ) ,$$

the lower quartile vector of the Euclidean matrix and the distance difference

$$( {NOR}_{c - 75 Q} , {DON}_{c - 75 Q} ) ,$$

the standard deviation vector of the Euclidean matrix and the distance difference

$$( {NOR}_{c - Std} , {DON}_{c - Std} ) ,$$

the deviation vector of the Euclidean matrix and the distance difference

$$( N O {R}_{c - p 2 p} , DO {N}_{c - p 2 p} ) ,$$

the l-th result

$$N O {R}_{c - Mean}^{l}$$

in NOR_{c-Mean} is obtained by averaging the Euclidean distances

$$N O {R}_{c}^{l} , N O {R}_{c + 1}^{l} , … , {NOR}_{c + f - 1}^{l}$$

of the class l waveform data in the Euclidean matrix vector within the c-th sliding window, therefore,

$$N O {R}_{c - Mean} = ( NO {R}_{c - Mean}^{1} , N O {R}_{c - M e a n}^{2} , N O {R}_{c - M e a n}^{3} ) ;$$

the l-th result

$${DON}_{c - Mean}^{l}$$

in DON_{c-Mean} is obtained by averaging the distance
differences

$${DON}_{c}^{l} , {DON}_{c + 1}^{l} , … , {DON}_{c + f - 1}^{l}$$

of the class l waveform data in the distance difference vector within the c-th sliding window, therefore,

$$D O {N}_{c - Mean} = ( DO {N}_{c - Mean}^{1} , D O {N}_{c - M e a n}^{2} , D O {N}_{c - M e a n}^{3} ) ;$$

other derived features after the second time sliding window processing can be determined based on their feature names, the corresponding computation process is performed on

$$N O {R}_{c}^{l} , N O {R}_{c + 1}^{l} , … , {NOR}_{c + f - 1}^{l} ,$$

and will not be described again here.

Therefore, the time series after the second time sliding window processing

$${T}^{″} = 〈 {H}_{1} , … , {H}_{c} , … , {H}_{{C}^{″}} 〉 , wherein$$

$${H}_{c} = 〈 \begin{matrix}{NOR}_{c - Mean} , {NOR}_{c - Max} , {NOR}_{c - Min} , {NOR}_{c - 25 Q} , \\ {NOR}_{c - 50 Q} , {NOR}_{c - 75 Q} , {NOR}_{c - Std} , {NOR}_{c - p 2 p} , \\ {DON}_{c - Mean} , {DON}_{c - Max} , {DON}_{c - Min} , {DON}_{c - 25 Q} , \\ {DON}_{c - 50 Q} , {DON}_{c - 75 Q} , {DON}_{c - Std} , {DON}_{c - p 2 p}\end{matrix} 〉 .$$

A schematic diagram of the time series after the two times sliding window processing is shown in FIG. 3(b).

(c) The time series after the sliding window processing are input into the neural network model, after processing, the neural network model outputs normal events as well as ventilator abnormal events (specifically, the types of patient-ventilator asynchronous events and their occurrence times); the types of ventilator abnormal events comprise ineffective effort, double triggering, auto-triggering, reverse triggering, flow starvation, overshooting, delayed cycling and premature cycling;
based on the output category results of the neural network model, the total number of patient-ventilator asynchronous and the index of patient-ventilator asynchronous can also be calculated, which can be calculated according to the existing methods and will not be described again here.

(2) In the physiological parameters data, the waveform data comprises:
photoplethysmogram waveform data and invasive arterial pressure waveform data;
the numerical data comprises: heart rate, blood pressure, blood oxygen saturation and body temperature; during the data labeling process for the physiological parameters data.

The shapes of the labeled electrocardiogram waveform data comprise: P wave, QRS complex, T wave, ST segment, PR interval, RR interval and QT interval of each ECG cycle waveform; the shapes of the labeled respiratory waveform data comprise: peaks and valleys of each respiratory cycle waveform; the shapes of the labeled photoplethysmogram waveform data comprises: peaks and valleys of each pulse cycle waveform; the shapes of the labeled invasive arterial pressure waveform data comprises: peaks and valleys of each arterial systolic-diastolic cycle waveform; the labeled contents can be configured to generate the report overview page and the report analysis page either directly or after statistics, computation and analysis.

The measurement values of the labeled numerical data comprise: parameters that are higher than the upper limit threshold of the corresponding numerical data or lower than the lower limit threshold of the corresponding numerical data; the labeled contents can be configured to generate the 24-hour physiological parameters data sub-page either directly or after statistics, computation and analysis.

The labeled abnormal events comprise: tachycardia, bradycardia, arrhythmia, cardiac arrest, atrial flutter, atrial fibrillation, ventricular flutter, ventricular fibrillation, atrioventricular block, ST-segment elevation/depression, high/low heart rate, high/low blood pressure, high/low body temperature and low blood oxygen saturation; the labeled contents can be configured to generate the report overview page, the report analysis page and the alarm events data sub-page either directly or after statistics, computation and analysis.

(3) In the blood gas analysis data, the numerical data comprises: partial pressure of oxygen, partial pressure of carbon dioxide, total carbon dioxide, potassium ion concentration, sodium ion concentration, bicarbonate ion concentration and pH value.

In the data labeling process for the blood gas analysis data,
the measurement values of the labeled numerical data comprise: parameters that are higher than the upper limit threshold of the corresponding numerical data or lower than the lower limit threshold of the corresponding numerical data; the labeled contents can be configured to generate the 24-hour blood gas analysis and laboratory diagnostic data sub-page either directly or after statistics, computation and analysis.

The labeled abnormal events comprise: high/low partial pressure of oxygen, high/low partial pressure of carbon dioxide, high/low total carbon dioxide, high/low potassium ion concentration, high/low sodium ion concentration, high/low bicarbonate ion concentration and high/low pH value. The labeled contents can be configured to generate the 24-hour blood gas analysis and laboratory diagnostic data sub-page either directly or after statistics, computation and analysis.

(4) In the laboratory diagnostic data, the numerical data comprises: white blood cell count, red blood cell count, platelet count, hemoglobin, hematocrit, blood glucose, prothrombin time and activated partial thrombin time.

In the data labeling process for the laboratory diagnostic data,
the measurement values of the labeled numerical data comprise: parameters that are higher than the upper limit threshold of the corresponding numerical data or lower than the lower limit threshold of the corresponding numerical data; the labeled contents can be configured to generate the 24-hour blood gas analysis and laboratory diagnostic data sub-page either directly or after statistics, computation and analysis.

The labeled abnormal events comprise: high/low white blood cell count, high/low red blood cell count, high/low hemoglobin, high/low hematocrit, high/low blood glucose, high/low platelet count, long/short prothrombin time, and long/short activated partial thrombin time. The labeled contents can be configured to generate the 24-hour blood gas analysis and laboratory diagnostic data sub-page either directly or after statistics, computation and analysis.

Other unexhausted mechanical ventilation therapy data and their data labeling are also easily conceived by a person skilled in the art based on the solution of the present embodiment, and therefore, also fall within the scope of protection of the present application.

Step S4: when a report generation instruction is received, generating a mechanical ventilation therapy data analysis report based on the medical data set with the data labeling.

For example, in step S4, retrieving the medical data set with the data labeling, performing the multi-dimensional data aggregation analysis to generate the analysis conclusion based on correlation, while various types of data are combined with the time labels and dimension labels and filled into the preset data analysis report framework to generate the mechanical ventilation therapy data analysis report, which is stored in the database. In addition, in order to further ensure the accuracy of the mechanical ventilation therapy data analysis report, manual review operation can be added, the mechanical ventilation therapy data analysis report after manual review is stored in the database.

It should be noted that the various types of data in the medical data set are computed, analyzed, and counted layer by layer to obtain data that meets the required contents and accuracy requirements in the preset framework of the analysis report, configured to generate the mechanical ventilation therapy data analysis report.

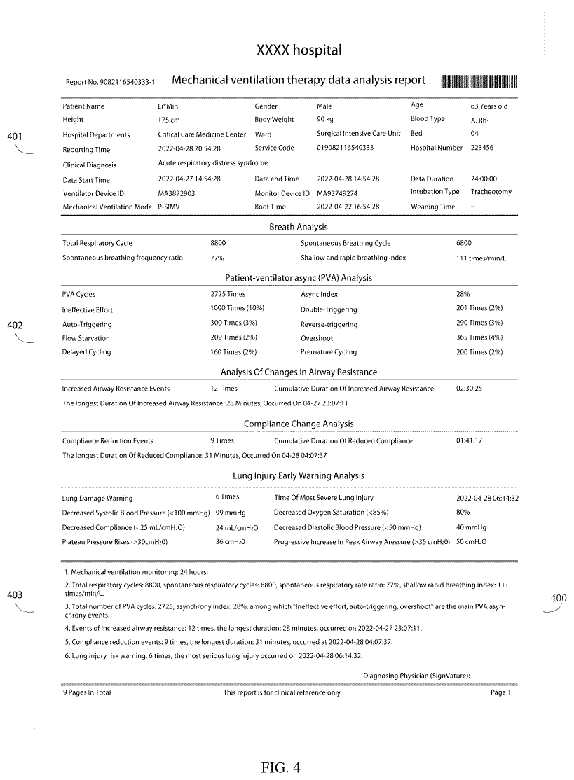

The reporting framework of the mechanical ventilation therapy data analysis report comprises a report overview page and a report analysis page; wherein as shown in FIG. 4, the report overview page 400 is divided into a basic information area 401, a mechanical ventilation data area 402, and an analysis conclusion area 403.

The basic information area 401 comprises at least patient information and key time point information; and further comprises the following options: the clinical diagnosis information, the ID of the device for outputting the mechanical ventilation therapy data as well as mechanical ventilation mode and setting information; for example, the patient information can comprise hospital information, basic patient information, ward beds information, hospital departments, etc.; the key time point information can comprise report time, data start time, data end time, device start up time, device shut down time, etc.,

The mechanical ventilation data area 402 comprises at least respiratory analysis results, patient-ventilator asynchronous events analysis results, airway resistance change analysis results, compliance change analysis results, and lung injury early warning analysis results; each of the analysis results is obtained based on the mechanical ventilation data and its data labeling; for example, the respiratory analysis results comprise: a total respiratory cycle, a spontaneous breathing cycle, a proportion of spontaneous breathing frequency and a rapid shallow breathing index.

Specifically, in the present embodiment, the total respiratory cycles, spontaneous breathing cycles and tidal volume labeled for the mechanical ventilation waveform data are statistically computed to obtain the number of total respiratory cycles, the number of spontaneous breathing cycles, the proportion of spontaneous breathing frequency, and the rapid shallow breathing index, configured to evaluate the possibility of the patient going offline. The data labeled for the mechanical ventilation patient-ventilator asynchronous events is statistical computed to obtain the total number of the patient-ventilator asynchronous events, the frequency of each type of asynchronous events is counted and its proportion is calculated. The compliance data labeled for the mechanical ventilation numerical data is computed to obtain the value and occurrence time of compliance reduction, and counts the number of times of compliance reduction, cumulative duration and the longest reduction duration within the preset time range. FIG. 4 shows an example of each analysis result in the mechanical ventilation data area, these example contents are the indicator data that clinicians are more concerned about and have high clinical value.

The analysis conclusion area 403 in FIG. 4 is configured to display the analysis descriptions and conclusions of the mechanical ventilation therapy data; wherein the analysis conclusion area can comprise the conclusion of the mechanical ventilation monitoring time measurement, the analysis conclusion of the respiratory (comprising the “rapid shallow breathing index”), the analysis conclusion of the patient-ventilator asynchronous events, the analysis conclusion of the airway resistance changes, the analysis conclusion of the compliance change, and the analysis conclusion of the lung injury early warning. Specifically, the various types of data in the mechanical ventilation data area are counted to obtain the two major categories of data such as normal interval and abnormal interval; the events are stratified according to the number of occurrence, the time of occurrence, the duration of the events in the abnormal interval, the type and the risk degree of the events in the abnormal interval, and are associated with the corresponding various types of analysis conclusion items to describe the state of the patient's mechanical ventilation therapy, generate analysis conclusions, and fill into the preset data analysis report framework. For example, the analysis conclusion of compliance change counts the number of occurrences of the events that meet the preset compliance reduction abnormal interval (<25 ml/cmH₂O) according to the labeled compliance reduction event data, and compares the duration of all the compliance change abnormal events to obtain the longest lasting compliance reduction event. Finally, the analytic conclusions were generated according to the number of occurrences of compliance reduction events, the longest lasting compliance reduction event and the time of occurrence, then filled into the analytic conclusion area 403.

The report analysis page is configured to display a detailed analysis report of one or more mechanical ventilation therapy data separately in graphical form.

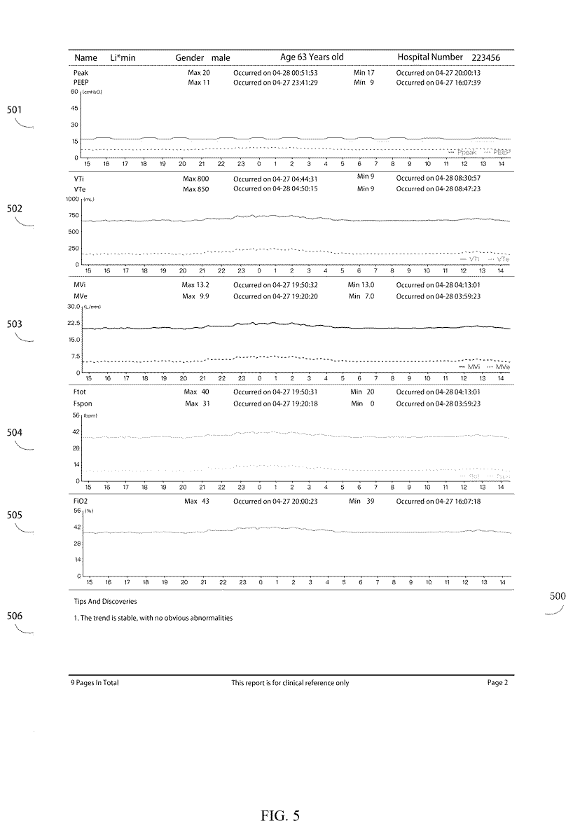

The report analysis page comprises a trend graph page 500, as shown in FIG. 5; the trend graph page comprises: a pressure trend analysis area 501, a volume trend analysis area 502, a ventilation trend analysis area 503, a trend analysis area of a respiratory frequency 504, a trend analysis area of an inhaled oxygen concentration 505, and a trend analysis description and conclusion area 506 of the mechanical ventilation data. Wherein the trend analysis description and conclusion area 506, configured to synthesize the trend direction of each trend analysis area to provide trend analysis descriptions and the corresponding conclusions. In addition, the contents of each trend analysis area are obtained based on the mechanical ventilation data and its data labeling, dividing into 24 number of hour data units by hour, calculating the average value of the parameters related with the report in each unit, and drawing the trend graph by plotting the average value of hourly parameters with a line.

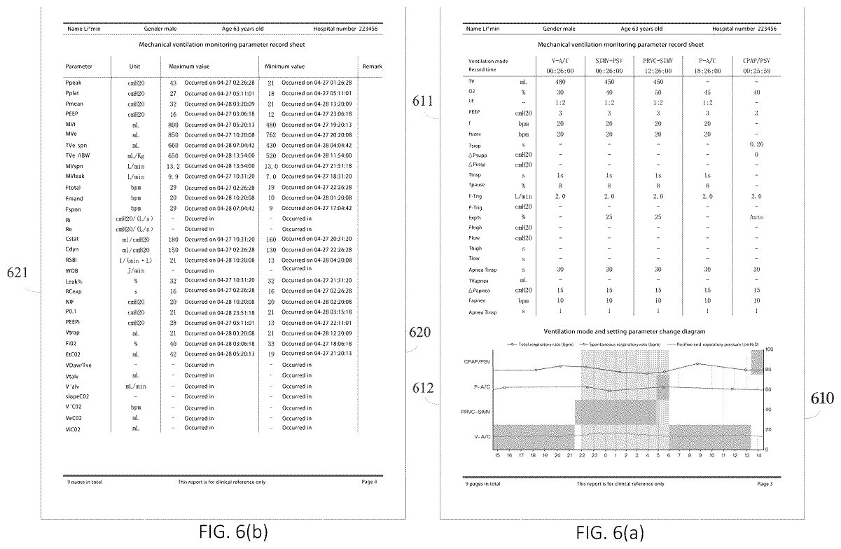

The report analysis page further comprises a data page, as shown in FIG. 6; the data page comprises at least: a mechanical ventilation setting parameters data sub-page 610, a mechanical ventilation monitoring parameters data sub-page 620; the data page further comprises the following optional data sub-pages: a 24-hour physiological parameters data sub-page 630, a 24-hour blood gas analysis and laboratory diagnostic data sub-page 640, and an alarm events data sub-page 650.

Wherein, the mechanical ventilation setting parameters data sub-page 610, as shown in FIG. 6(a), comprising a mechanical ventilation setting parameters record sheet 611, ventilation modes of mechanical ventilation and a change trend graph of setting parameters 612.

The mechanical ventilation monitoring parameters data sub-page 620, as shown in FIG. 6(b); comprising a mechanical ventilation monitoring parameters record sheet 621; the contents of the mechanical ventilation setting parameters data sub-page and the mechanical ventilation monitoring parameters data sub-page 620 are obtained based on the mechanical ventilation data and its data labeling.

The 24-hour physiological parameters data sub-page 630, as shown in FIG. 6(c); comprising a 24-hour vital signs monitoring record sheet 631 and a heart rate/respiratory rate/blood pressure trend graph 632; the contents of the 24-hour physiological parameters data sub-page 630 are obtained based on the physiological parameters data and its data labeling.

The 24-hour blood gas analysis and laboratory diagnostic data sub-page 640, as shown in FIG. 6(d); comprising a 24-hour blood gas analysis monitoring record sheet 641, a partial pressure of oxygen/partial pressure of carbon dioxide/bicarbonate trend graph 642, and an laboratory diagnostic data sheet 643; the contents of the 24-hour blood gas analysis and laboratory diagnostic data sub-page 640 are obtained based on the blood gas analysis data, the laboratory diagnostic data, and their data labeling.

The alarm events data sub-page 650, as shown in FIG. 6(e); comprising an alarm events record sheet 651, a 24-hour alarm events distribution diagram 652, and an alarm events comprehensive analysis sheet 653. The contents of the alarm events data sub-page 650 are obtained based on the abnormal events labeled by the mechanical ventilation therapy data.

The extended image data sub-page 660, as shown in FIG. 6(e); configured to display the image data. For example, the image data can comprise an ultrasound image examination area 661, a CT image examination area 662, and a DR image examination area 663.

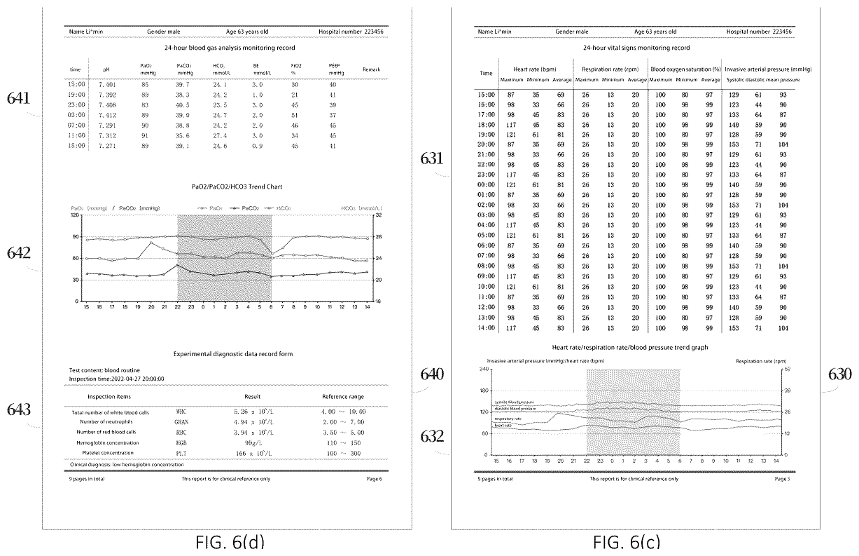

The report analysis page further comprises a graphic page 700, as shown in FIG. 7; the graphic page is divided into a mechanical ventilation waveform data area 701, a physiological parameters waveform data area 702, and a mechanical ventilation loop chart area 703.

Wherein, the mechanical ventilation waveform data area 701 comprises waveform graphs of continuous normal mechanical ventilation data and waveform graphs of abnormal mechanical ventilation events data, and the waveform graphs have waveform measurement values, labeling of each waveform cycle and labeled types of the abnormal events attached to them.

The physiological parameter waveform data area 702 comprises waveform graphs of continuous physiological parameters data synchronized with the mechanical ventilation data waveform graphs, and the waveform graphs have waveform measurement values, labeling of each waveform cycle and labeled types of the abnormal events. The contents of the physiological parameter waveform data area are obtained based on the physiological parameters data and its data labeling.

The mechanical ventilation loop chart area 703 comprises a pressure-volume loop chart and a flow-volume loop chart drawn by the combination of the pressure, volume, and flow data within a mechanical ventilation event cycle, and highlighting the abnormal loop chart in a superimposed manner; the contents of the mechanical ventilation waveform data area and the mechanical ventilation loop chart area are obtained based on the mechanical ventilation data and its data labeling; for example, using the mechanical ventilation data to plot the normal mechanical ventilation data waveform graphs, the abnormal mechanical ventilation event data waveform graphs, and the mechanical ventilation loop charts, and superimposing the loop charts formed by each mechanical ventilation, highlighting the loop charts with changes in morphology, and labeling the time of occurrence and duration.

The above graphic page can be laid out according to the order of the mechanical ventilation waveform data area and the physiological parameters waveform data area; the order of the mechanical ventilation waveform data and the physiological parameters waveform data can also be reorganized according to the data usage habits and thinking ways of clinician.

It should be emphasized that the mechanical ventilation therapy data analysis report is a multi-types, multi time series summarized data set, which provides summarized analyses and conclusions of the key indexes data of the mechanical ventilation therapy, as well as various key indexes data, abnormal events data, and evolutionary trends, comprising physiological parameters data, blood gas analysis data and laboratory diagnostic data during the therapy; and provides waveform graphic data that reflects the state of ventilator's function, the state of the patient/ventilator interaction, the state of respiratory mechanics, the state of physiological parameters, the waveform graphic data of all kinds of event state; can also provide the image data of lung, heart, and abdominal diaphragm. Based on the mechanical ventilation therapy data analysis report, doctors and nurses can quickly grasp the comprehensive mechanical ventilation therapy data, disease status and evolution trend, evaluate the effectiveness of the treatment strategy, provide timely intervention measures to reduce lung injury and other complications, improved the efficiency of data processing and the quality of mechanical ventilation therapy, promoted the recovery of patients, which has wide applicability.

In actual application, in order to extend the application scenes of the solution in the present embodiment, the report generation instruction can be a customized report generation instruction; the customized report generation instruction comprises one or more customized parameters, configured to generate the report overview page and the report analysis page matching the customized parameters; the customized parameters comprise the trend graph page, the data page, or the graph page. For example, when only the trend graph page is comprised in the customized parameters, the report analysis page in the generated mechanical ventilation therapy data analysis report also comprises only the trend graph page and no longer comprises the data page and the graphic page. The generation of each item in the mechanical ventilation therapy data analysis report can be referred to the above description and will not be described again here.

In addition, the method further comprises:
Step S5: in the data labeling process, a first aid instruction is issued when one or more abnormal events of ventilators stop working, asphyxia, increasing airway pressure, cardiac arrest, tachycardia, bradycardia, ventricular flutter, ventricular fibrillation and hypotension (shock) occur, the first aid instruction carries the information about the abnormal events that occur.

Step S6: the method further comprises:
in the data labeling process, a ventilator parameters adjustment instruction is issued when one or more abnormal events of patient-ventilator asynchronous event, high airway pressure, low airway pressure, high positive end expiratory pressure, low positive end expiratory pressure, high minute ventilation, low minute ventilation, high tidal volume, low tidal volume, high respiratory rate, low respiratory rate, high oxygen concentration and low oxygen concentration occur, the ventilator parameters adjustment instruction carries the information about the abnormal events that occur.

In addition, step S7: the method further comprises:
while receiving the mechanical ventilation therapy data in real time, streaming media data of the treatment scenes is also received in real time (for example, the streaming media data can be collected by bedside audio video devices); is live broadcast in real time of the waveform data, the numeric data, and the treatment scenes in the mechanical ventilation therapy data of the medical data set, as well as the mechanical ventilation therapy data analysis reports of some or all patients in one or more wards. For example, the clinical data terminal devices can live broadcast the mechanical ventilation waveform data, the numeric data, and the streaming media data of the medical data set. When the data is derived from a plurality of patients in a plurality of wards, the data and treatment scenes of patients in the plurality of wards are displayed in a visualized way; furthermore, the streaming media data can be associated with the medical data set, all of which are stored into the database.

The present embodiment provides a method of mechanical ventilation therapy data management, doctors and nurses remotely and centrally can manage the mechanical ventilation therapy data business of a plurality of wards in a visualized way, process a huge amount of heterogeneous data generated by a plurality of devices into data with standardized data structure to generate the mechanical ventilation therapy data analysis reports. Doctors and nurses are able to observe and analyze the real-time data, events data, data analysis reports, treatment scenes of the mechanical ventilation therapy of patients in a plurality of wards, comprising the laboratory diagnostic data and the image data from the clinical information systems, on a single clinical data terminal device, master the current and past mechanical ventilation therapy status and disease evolution trend, discover complications at an early stage, and provide timely intervention measures to improve the quality of mechanical ventilation therapy and work efficiency, reduce the labor intensity of doctors and nurses, solve the difficulties caused by huge multi-source heterogeneous data to the clinical application, and has wide applicability.

### Embodiment 2

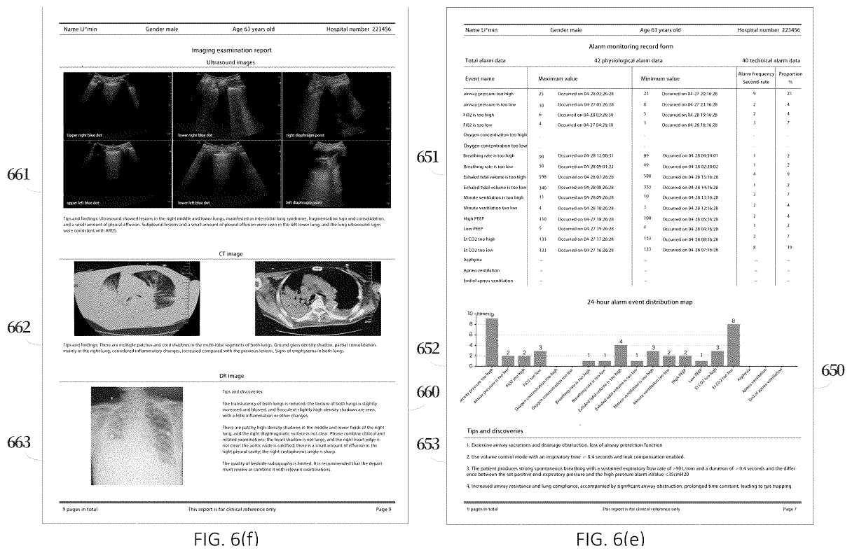

The embodiment of the present application discloses a system of mechanical ventilation therapy data management, as shown in FIG. 8, the system comprises an application software system 801, clinical data terminal devices 802 and a database server 803.

Wherein, the application software system 801 comprises a data acquisition and standardization unit, a data cleaning unit, a data labeling unit and a report generation unit.

Specifically, the data acquisition and standardization unit is configured to put the patient information and the real-time received mechanical ventilation therapy data into the standard data structure object respectively to form standardized data; the mechanical ventilation therapy data comprises ventilator data.

The data cleansing unit is configured to perform data cleansing on the standardized data to obtain a medical data set.

The data labeling unit is configured to perform data labeling on the mechanical ventilation therapy data in the medical data set; the data labeling comprises: marking amplitudes, frequencies, and shapes of waveform data, marking measurement values of numerical data, and marking abnormal events.

The report generation unit is configured to generate a mechanical ventilation therapy data analysis report based on the medical data set with the data labeling when a report generation instruction is received.

The clinical data terminal devices 802 are configured to issue the report generation instructions, and further configured to receive and display the mechanical ventilation therapy data analysis reports.

The database server 803 is configured to store the standardized data, the medical data sets, and the mechanical ventilation therapy data analysis reports in partitions.

The other data comprised in the mechanical ventilation therapy data, the data labeling process and the mechanical ventilation therapy data analysis report generating process have been described in detail in the embodiment 1, and will not be described again here.

In addition, in the present embodiment, in the data labeling process of the data labeling unit, a first aid instruction is issued to the clinical data terminal devices when one or more abnormal events of ventilators stop working, asphyxia, increasing airway pressure, cardiac arrest, tachycardia, bradycardia, ventricular flutter, ventricular fibrillation and hypotension occur, the first aid instruction carries the information about the abnormal events that occur; at this time, the clinical data terminal devices 802 are further configured to receive and respond to the first aid instructions. For example, the clinical data terminal devices 802 can issue an abnormal alarm in respond to the first aid instruction, to draw the attention of the medical staff, and can also display the abnormal events information via the clinical data terminal devices.

In the data labeling process of the data labeling unit, a ventilator parameters adjustment instruction is issued to the clinical data terminal devices 802 when one or more abnormal events of patient-ventilator asynchronous event, high airway pressure, low airway pressure, high positive end expiratory pressure, low positive end expiratory pressure, high minute ventilation, low minute ventilation, high tidal volume, low tidal volume, high respiratory rate, low respiratory rate, high oxygen concentration and low oxygen concentration occur, the ventilator parameter adjustment instruction carries the information about the abnormal events that occur; at this time, the clinical data terminal devices 802 are further configured to receive and respond to the ventilator parameters adjustment instructions, the ventilator parameter adjustment instruction can be responded by issuing an abnormal alarm to draw the attention of the medical staff, and can also display the abnormal events information via the clinical data terminal devices 802, and can also provide a suggestion of the ventilator parameter adjustment method for the current abnormal event.

The application software system 801 further comprises a streaming media management unit, the streaming media management unit is configured to receive streaming media data of the treatment scenes in real time via the bedside audio video devices; the streaming media management unit can also manage the operation of the bedside audio video devices, comprising streaming media service management, equipment scheduling, consultation management, ward round management, and live broadcasting, storing, reviewing, and retrieving of streaming media data. At this time, the database server 803 is further configured to store the streaming media data in partitions and associate the streaming media data with the medical data sets and the mechanical ventilation therapy data analysis reports; the clinical data terminal devices 802 are further configured to live broadcast in real time of the waveform data, the numeric data, and the treatment scenes in the mechanical ventilation therapy data of the medical data set as well as the mechanical ventilation therapy data analysis reports of some or all patients in one or more wards.

As shown in FIG. 8, the system of mechanical ventilation therapy data management can further comprise a multi-mode gateway 804 and a master control server 805; wherein the multi-mode gateway is configured to connect the ventilator devices 807, the multi-parameter monitors 808, the bedside audio video devices 809, the clinical information systems 810 and the clinical data terminal devices 802 from different manufacturers in each ward of the hospital through the network; the multi-mode gateway 804 is embedded with a variety of communication protocols, comprising at least Transmission Control Protocol (TCP)/Internet Protocol (IP), User Datagram Protocol (UDP), Recommended Standard 232/485 (RS 232/485), Representational State Transfer Application Programming Interface (RESTful API), Wi-Fi (WIreless FIdelity), Message Queuing Telemetry Transport (MQTT), and Health Level seven (HL7), configured to realize data communication between the ventilator devices 807, the multi-parameter monitors 808, the bedside audio video devices 809, the clinical information systems 810 and the data acquisition and standardization unit, and configured to realize the data communication between the application software system 801 and the clinical data terminal devices 802. The master control server 805, embedded with a multi-task operating system and an application software system 801 running on the operating system, configured to control the operation of the application software system 801.

The database server 803, consisting of a plurality of servers to form a database cluster, is configured to store various types of data and files for the system, in addition, is further configured to perform real-time analysis and processing, multi-dimensional retrieval and query, multi-user network operation, and database disaster recovery management. It should be noted that the database server 803 can use a plurality of servers to form the database cluster, wherein one server is set as the master node and the rest are set as sub-nodes, as a copy of the master node, to divert the business pressure of the master node server, when the master node encounters an exception during operation, the sub-nodes can adaptively switch to be the master node, which can improve the security and high availability of the database, and meet the storage of massive mechanical ventilation therapy data, data analysis report files, streaming media data for the system, database cluster can be dynamically expanded or migrated to the cloud more conveniently.

The clinical data terminal devices 802, comprising at least desktop computers and desktop devices with computing capabilities, can be configured to perform data communication with the system and display the mechanical ventilation therapy data and status of the patients; common clinical data terminal devices such as handheld terminal devices 811 held by doctors and nurses, comprising at least laptop computers and smartphones, can be configured to display the mechanical ventilation therapy data and status of the patients.

The bedside audio video devices 809, comprising at least video monitoring devices and audio video mobile carts, can be configured to display the patient treatment scenes on the screen of the clinical data terminal devices.

In the present embodiment, the various units in the application software system 801 cooperate with each other to jointly complete the functions of data acquisition, data cleaning, data labeling and report generation, which can realize multi-dimensional analysis and comprehensive judgment of the patient's mechanical ventilation therapy process, thereby predicting the disease evolution trend, facilitating doctors to make intervention measures, which effectively improves the quality of mechanical ventilation therapy and work efficiency, reduces labor intensity of doctors and nurses. The screen of the clinical data terminal devices 802 can be a single screen or a combination of screens, the doctors and nurses holding the data terminal devices 811 can perform seamless monitoring and management of the mechanical ventilation therapy patients in any situation.

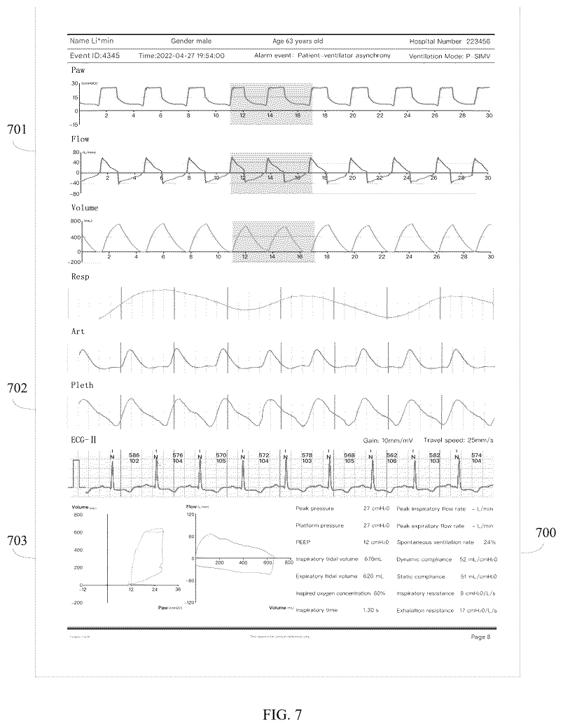

Preferably, FIG. 9 of the present embodiment shows an implementation example displaying the patient in a visualized way, wherein the clinical data terminal devices 901 can receive the mechanical ventilation therapy data and the streaming media data of the patients 902 in a plurality of wards in real time, dynamically display the waveform data, the numerical data and the treatment scenes of the patients in the plurality of wards in a visualized way to remotely and centrally manage their mechanical ventilation therapy data business of the patients in the plurality of wards.

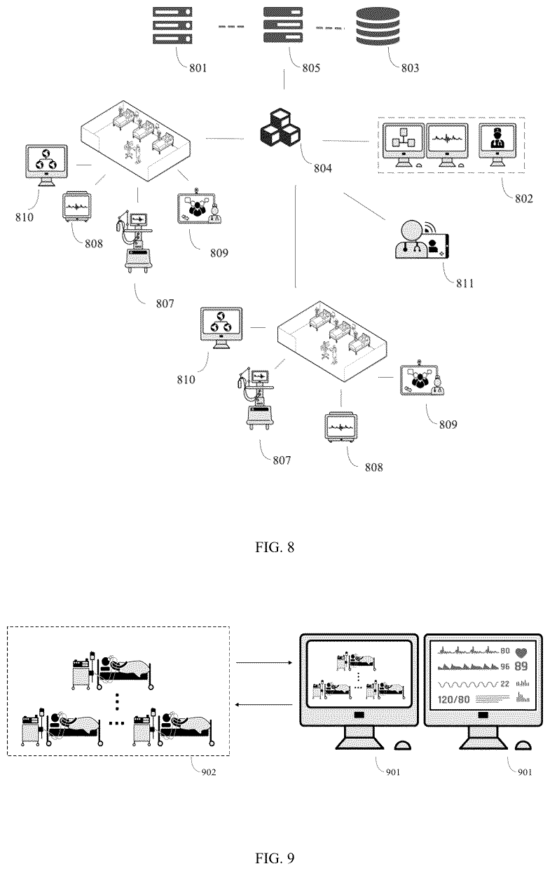

Preferably, a ward management panel 1000 provided by the clinical data terminal device screen as shown in FIG. 10, so as to centrally manage the mechanical ventilation data business of patients in a plurality of wards, can display the patient's mechanical ventilation therapy data and disease evolution trend in a visualized way, observe and analyze a variety of data, events data and treatment scenes, output clinical diagnostic information, as well as medical advice, nursing information and business instructions, wherein the business instructions at least comprise first aid instructions, ventilator parameters adjustment instructions, and generating analysis report instructions. The ward management panel is set up with content window comprising a functional operation area 1001, a ward management area 1002, a patient data management area 1003, and a clinical application management area 1004, as well as functional buttons, which can allow selecting all or a certain patient in a plurality of wards to observe the patient's real-time data, events data, historical data, clinical medical advice and nursing information, and treatment scenes, analyzing the mechanical ventilation therapy, issuing business instructions or medical advice. Specifically, the functional operation area 1001 can be equipped with function buttons, configured to switch between panels of the clinical data terminal devices, and also configured to select a specified patient; the ward management area 1002 can be configured to display and retrieve the patients information, wards information and reports information; the patient data management area 1003 can be configured to display and retrieve the mechanical ventilation therapy data of the patients; the clinical application management area 1004 cab be configured to display and retrieve the patient's streaming media data and mechanical ventilation therapy data, issue business instructions or medical advice.

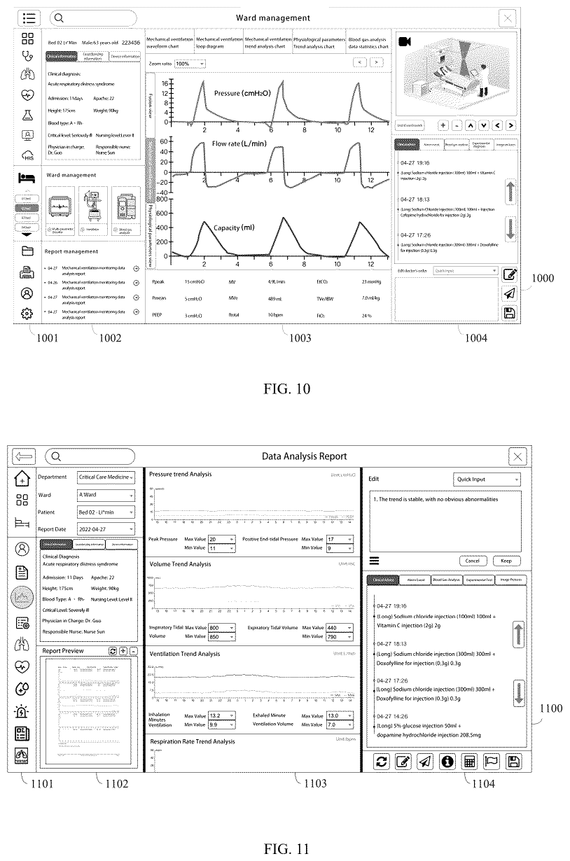

Preferably, FIG. 11 shows an implementation example of a data analysis report panel 1100 of a clinical data terminal device provided in the embodiment 2 of the present application; the clinical data terminal device screen provides the data analysis report panel to visually display the mechanical ventilation analysis reports of all or a certain patient in a plurality of wards, thereby performing review, network distribution, output printing, and database storage for the mechanical ventilation data analysis reports according to the business instructions or the manual review requests automatically prompted by the medical advice appointment time. The data analysis report panel is set up with content window comprising a functional operation area 1101, a ward management area 1102, an analysis report area 1103, a clinical medical advice area 1104, and functional buttons to centrally manage the mechanical ventilation data analysis report business of a plurality of wards, analyze the reasons for data changes in conjunction with the clinical treatment medical advice information, perform comparative analysis on all the previous mechanical ventilation therapy data analysis reports of a certain patient to determine the disease evolution trend, and trigger the printer to output the mechanical ventilation therapy data analysis reports. Specifically, the function operation area 1101 is equipped with function buttons, configured to switch between data analysis panels of the clinical data terminal devices, and further configured to perform management operations on the reports; the ward management area 1102 is configured to display and retrieve the patients information, the wards information and the reports information, and further configured to select a specified patient or a report; the analysis report area 1103 is configured to display and retrieve the patients' data analysis reports and display them in a visualized way; the clinical medical advice area 1104 is configured to display and retrieve the patients' mechanical ventilation therapy data, edit report contents and trigger print commands.

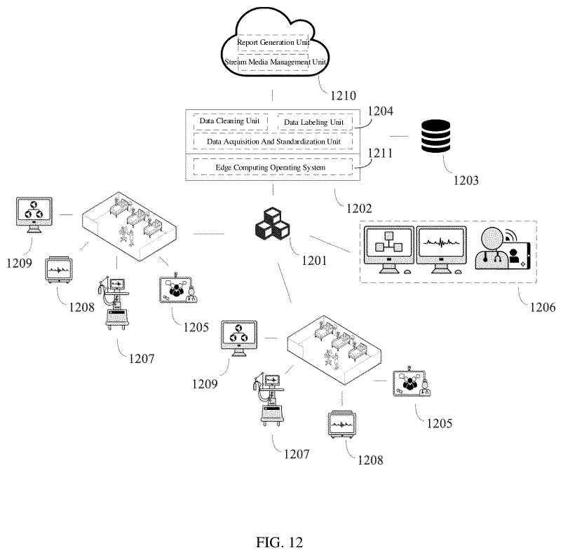

To further extend the scalability and flexibility of the system of mechanical ventilation therapy data management in the present embodiment, for example, the system of mechanical ventilation therapy data management can be deployed under a cloud computing architecture, i.e., each functional unit in the system of mechanical ventilation therapy data management is deployed under a cloud computing architecture; or some of the functional units in the system of mechanical ventilation therapy data management are deployed on a cloud platform, for example, the report generation unit and streaming media management unit, etc., which are configured to realize various business functions of the system, comprise cloud edge collaborative live broadcast in real time of the waveform data, the numeric data, and the treatment scenes; other functional units are deployed on edge computing nodes, such as data acquisition and standardization unit, data cleaning unit, data labeling unit, etc., which are configured to perform data cleaning and data standardization at the data source, as well as perform data labeling and events labeling, providing support for cloud edge collaboration, cloud platform data storage management, fast multi-dimensional data retrieval, and data updating. As shown in FIG. 12, the system comprises a multi-mode gateway 1201, a master control server 1202, a database server 1203, an application software system 1204, bedside audio video devices 1205, clinical data terminal devices 1206; the multi-mode gateway 1201 connects the bedside audio video devices 1205, the ventilators 1207, the multi-parameter monitors 1208, the clinical information systems 1209 and the clinical data terminal devices 1206 from different manufacturers in each ward of the hospital through the network, and establishes a connection with the cloud platform 1210.

The system of mechanical ventilation therapy data management in the present embodiment can also connect to a third-party medical data service, entrusting the real-time monitoring, data analysis and processing of the complex and tedious mechanical ventilation therapy data to a third-party for management, so that the doctors and nurses are able to focus on the clinical medical issues of the mechanical ventilation therapy, to further reduce the work pressure of the doctors and nurses, and to improve the quality of the mechanical ventilation therapy and the work efficiency.

The above method and system embodiments improve the existing clinical application method of the mechanical ventilation therapy data, doctors and nurses can remotely and centrally manage the patients in a plurality of wards, quickly grasp comprehensive mechanical ventilation data and the disease evolution trend, which solves the difficulties caused by huge multi-source heterogeneous data to the clinical application, reduces the work pressure of doctors and nurses, improves the quality of mechanical ventilation therapy, and has wide applicability.

The present embodiment can reduce hardware costs and business pressure, performs data preprocessing at the data source, reduce the delay of network data communication and cloud platform processing effectively, can flexibly determine the scale of user data storage, for example, limiting the data files to be saved for a period of three months, reduce the pressure and cost of storing the mechanical ventilation therapy data. When there is a malfunction in the external network, the edge computing nodes can complete the processing of the mechanical ventilation therapy data analysis reports independently.

A person skilled in the art can understand that all or part of the process of implementing the methods of the above embodiments may be implemented by related hardware according to an instruction from a computer program, and the program may be stored in a computer-readable storage medium, wherein the computer-readable storage medium is a magnetic disc, an optical disc, a read-only memory, a random access memory and so on.

The above are merely preferable particular embodiments of the present application, and the protection scope of the present application is not limited thereto. All of the variations or substitutions that a person skilled in the art can easily envisage within the technical scope disclosed by the present application should fall within the protection scope of the present application.

1. A method of mechanical ventilation therapy data management, wherein the method comprises:
putting patient information and real-time received mechanical ventilation therapy data into a standard data structure object respectively, to form standardized data; the mechanical ventilation therapy data comprises ventilator data;
performing data cleaning on the standardized data to obtain a medical data set;
performing data labeling on the mechanical ventilation therapy data in the medical data set; the data labeling comprises: marking amplitudes, frequencies, and shapes of waveform data, marking measurement values of numerical data, and marking abnormal events;
when a report generation instruction is received, generating a mechanical ventilation therapy data analysis report based on the medical data set with the data labeling.

2. The method of mechanical ventilation therapy data management according to claim 1, wherein the mechanical ventilation therapy data further comprises one or more of the following data: physiological parameters data, blood gas analysis data, laboratory diagnostic data, imaging data, clinical diagnosis and treatment information, and the ID of the device for outputting the mechanical ventilation therapy data; wherein,
the physiological parameters data is output by multi-parameter monitors;
the patient information, the blood gas analysis data, the laboratory diagnostic data, the imaging data, and the clinical diagnosis and treatment information are all output by a clinical information system;
the mechanical ventilation data is output by ventilator devices.

3. The method of mechanical ventilation therapy data management according to claim 2, wherein in the mechanical ventilation data, the waveform data comprises: mechanical ventilation pressure waveform data, volume waveform data, flow waveform data and end-tidal carbon dioxide waveform data; the numerical data comprises: airway resistance, work of breathing, compliance, ventilation parameters and respiratory rate;
in the data labeling process for the mechanical ventilation data,
the shapes of the labeled waveform data comprise: beginning of inspiratory phase, inspiratory phase, end of inspiratory phase, beginning of expiratory phase, expiratory phase, end of expiratory phase, peak airway pressure, and peak expiratory flow rate of each mechanical ventilation waveform, as well as inspiratory baseline, expiratory plateau height, beginning and end of expiratory plateau of the end-tidal carbon dioxide waveform;
the labeled abnormal events comprise: patient-ventilator asynchronous event, high/low airway pressure, high/low positive end-expiratory pressure, high/low expired tidal volume, high/low minute ventilation, high/low inspired oxygen concentration, high/low oxygen concentration, high/low respiratory rate, high/low partial pressure of end-tidal carbon dioxide, high/low tidal volume, ventilators stop working, asphyxia and increasing airway pressure.

4. The method of mechanical ventilation therapy data management according to claim 3, wherein in the physiological parameters data, the waveform data comprises: electrocardiogram waveform data, respiratory waveform data, photoplethysmogram waveform data and invasive arterial pressure waveform data; the numerical data comprises: heart rate, blood pressure, blood oxygen saturation and body temperature;
in the data labeling process for the physiological parameters data,
the shapes of the labeled electrocardiogram waveform data comprise: P wave, QRS complex, T wave, ST segment, PR interval, RR interval and QT interval of each ECG cycle waveform;
the shapes of the labeled respiratory waveform data comprise: peaks and valleys of each respiratory cycle waveform;
the shapes of the labeled photoplethysmogram waveform data comprise: peaks and valleys of each pulse cycle waveform;
the shapes of the labeled invasive arterial pressure waveform data comprise: peaks and valleys of each arterial systolic-diastolic cycle waveform;
the labeled abnormal events comprise: tachycardia, bradycardia, arrhythmia, cardiac arrest, atrial flutter, atrial fibrillation, ventricular flutter, ventricular fibrillation, atrioventricular block, ST-segment elevation/depression, high/low heart rate, high/low blood pressure, high/low body temperature and low blood oxygen saturation.

5. The method of mechanical ventilation therapy data management according to claim 4, wherein, in the blood gas analysis data, the numerical data comprises: partial pressure of oxygen, partial pressure of carbon dioxide, total carbon dioxide, potassium ion concentration, sodium ion concentration, bicarbonate ion concentration and pH value;
in the data labeling process for the blood gas analysis data,
the labeled abnormal events comprise: high/low partial pressure of oxygen, high/low partial pressure of carbon dioxide, high/low total carbon dioxide, high/low potassium ion concentration, high/low sodium ion concentration and high/low pH value.

6. The method of mechanical ventilation therapy data management according to claim 5, wherein in the laboratory diagnostic data, the numerical data comprises: white blood cell count, red blood cell count, platelet count, hemoglobin, hematocrit, blood glucose, prothrombin time and activated partial thrombin time;
in the data labeling process for the laboratory diagnostic data,
the labeled abnormal events comprise: high/low white blood cell count, high/low red blood cell count, high/low hemoglobin, high/low hematocrit, high/low blood glucose, high/low platelet count, long/short prothrombin time, and long/short activated partial thrombin time.

7. The method of mechanical ventilation therapy data management according to claim 6, wherein the reporting framework of the mechanical ventilation therapy data analysis report comprises a report overview page and a report analysis page; wherein the report overview page is divided into a basic information area, a mechanical ventilation data area, and an analysis conclusion area;
the basic information area comprises at least the patient information and key time point information; and further comprises the following options: the clinical diagnosis information, the ID of the device for outputting the mechanical ventilation therapy data as well as mechanical ventilation mode and setting information;
the mechanical ventilation data area comprises at least respiratory analysis results, patient-ventilator asynchronous events analysis results, airway resistance change analysis results, compliance change analysis results, and lung injury early warning analysis results;
each of the analysis results of the mechanical ventilation data area is obtained based on the mechanical ventilation data and its data labeling; the respiratory analysis results comprise a rapid shallow breathing index;
the analysis conclusion area is configured to display the analysis descriptions and conclusions of the mechanical ventilation therapy data;
the report analysis page is configured to display a detailed analysis report of one or more mechanical ventilation therapy data separately in graphical form.

8. The method of mechanical ventilation therapy data management according to claim 7, wherein the report analysis page comprises a trend graph page; the trend graph page comprises:
trend analysis areas of a pressure, volume, and ventilation volume of the mechanical ventilation data;
a trend analysis area of a respiratory frequency;
a trend analysis area of an inhaled oxygen concentration; and
a trend analysis description and conclusion area is configured to synthesize the trend direction of each trend analysis area, to provide trend analysis descriptions and corresponding conclusions;
the contents of each trend analysis area in the trend graph page are obtained based on the mechanical ventilation data and its data labeling.

9. The method of mechanical ventilation therapy data management according to claim 8, wherein the report analysis page further comprises a data page;
the data page comprises at least:
a mechanical ventilation setting parameters data sub-page comprises: a mechanical ventilation setting parameters record sheet, ventilation modes of mechanical ventilation and a change trend graph of setting parameters;
a mechanical ventilation monitoring parameters data sub-page comprises a mechanical ventilation monitoring parameters record sheet;
the contents of the mechanical ventilation setting parameters data sub-page and the mechanical ventilation monitoring parameters data sub-page are obtained based on the mechanical ventilation data and its data labeling;
the data page further comprises the following optional data sub-pages:
a 24-hour physiological parameters data sub-page comprises a 24-hour vital signs monitoring record sheet and a heart rate/respiratory rate/blood pressure trend graph; the contents of the 24-hour physiological parameters data sub-page are obtained based on the physiological parameters data and its data labeling;
a 24-hour blood gas analysis and laboratory diagnostic data sub-page comprises a 24-hour blood gas analysis monitoring record sheet, a partial pressure of oxygen/partial pressure of carbon dioxide/bicarbonate trend graph, and a laboratory diagnostic data sheet;
the contents of the 24-hour blood gas analysis and laboratory diagnostic data sub-page are obtained based on the blood gas analysis data, the laboratory diagnostic data, and their data labeling;
an alarm events data sub-page comprises: an alarm events record sheet, a 24-hour alarm events distribution diagram, and an alarm events comprehensive analysis sheet;
the alarm events data sub-page is obtained based on the abnormal events labeled by the mechanical ventilation therapy data;
an extended image data sub-page is configured to display the image data.

10. The method of mechanical ventilation therapy data management according to claim 9, wherein, the report analysis page further comprises a graphic page; the graphic page is divided into a mechanical ventilation waveform data area, a mechanical ventilation loop chart area, and a physiological parameters waveform data area; wherein
the mechanical ventilation waveform data area comprises waveform graphs of continuous normal mechanical ventilation data and waveform graphs of abnormal mechanical ventilation events data;
the mechanical ventilation loop chart area comprises a pressure-volume loop chart and a flow-volume loop chart drawn by the combination of the mechanical ventilation pressure, volume, and flow data;
the contents of the mechanical ventilation waveform data area and the mechanical ventilation loop chart area are obtained based on the mechanical ventilation data and its data labeling;
the physiological parameters waveform data area comprises waveform graphs of continuous physiological parameters data synchronized with the mechanical ventilation data waveform graphs;
the content of the physiological parameters waveform data area is obtained based on the physiological parameters data and its data labeling.

11. The method of mechanical ventilation therapy data management according to f-claims 7-10, wherein, the report generation instruction is a customized report generation instruction;
the customized report generation instruction comprises one or more customized parameters, is configured to generate the report overview page, and the report analysis page matching the customized parameters;
the customized parameters comprise the trend graph page, the data page, or the graphic page.

12. The method of mechanical ventilation therapy data management according to claim 7, wherein the method further comprises:
in the data labeling process, a first aid instruction is issued when one or more abnormal events of ventilators stop working, asphyxia, increasing airway pressure, cardiac arrest, tachycardia, bradycardia, ventricular flutter, ventricular fibrillation and hypotension occur, the first aid instruction carries the information about the abnormal events that occur;
in the data labeling process, a ventilator parameters adjustment instruction is issued when one or more abnormal events of patient-ventilator asynchronous events, high airway pressure, low airway pressure, high positive end expiratory pressure, low positive end expiratory pressure, high minute ventilation, low minute ventilation, high tidal volume, low tidal volume, high respiratory rate, low respiratory rate, high oxygen concentration and low oxygen concentration occur, wherein the ventilator parameters adjustment instruction carries the information about the abnormal events that occur.

13. (canceled)

14. The method of mechanical ventilation therapy data management according to claim 1, wherein, in the standard data structure object, a corresponding data structure object is configured for each item of the mechanical ventilation therapy data and the patient information respectively;
each mechanical ventilation therapy data decoupled and the patient information are placed into the corresponding data structure object to form standardized data.

15. (canceled)

16. A system of mechanical ventilation therapy data management, wherein, the system comprises an application software system, clinical data terminal devices and a database server; wherein,
the application software system comprises:
a data acquisition and standardization unit, configured to put the patient information and the real-time received mechanical ventilation therapy data into the standard data structure object respectively to form standardized data; the mechanical ventilation therapy data comprises ventilator data;
a data cleaning unit is configured to perform data cleaning on the standardized data to obtain a medical data set;
a data labeling unit is configured to perform data labeling on the mechanical ventilation therapy data in the medical data set;
the data labeling comprises: marking amplitudes, frequencies, and shapes of waveform data, marking measurement values of numerical data, and marking abnormal events;
a report generation unit is configured to generate a mechanical ventilation therapy data analysis report based on the medical data set with the data labeling when a report generation instruction is received;
the clinical data terminal devices are configured to issue the report generation instructions, and further configured to receive and display the mechanical ventilation therapy data analysis reports;
the database server is configured to store the standardized data, the medical data sets, and the mechanical ventilation therapy data analysis reports in partitions.

17.-29. (canceled)

30. The system of mechanical ventilation therapy data management according to claim 16, wherein the application software system further comprises a streaming media management unit, the streaming media management unit is configured to receive streaming media data of the treatment scenes in real time; the streaming media data is output by bedside audio video devices;
the database server is further configured to store the streaming media data in partitions and associate the streaming media data with the medical data sets and the mechanical ventilation therapy data analysis reports;
the clinical data terminal devices are further configured to live broadcast in real time of the waveform data, the numeric data, and the treatment scenes in the mechanical ventilation therapy data of the medical data set as well as the mechanical ventilation therapy data analysis reports of some or all patients in one or more wards.

31. The system of mechanical ventilation therapy data management according to claim 16, wherein the system further comprises a multi-mode gateway and a master control server; wherein
the multi-mode gateway is configured to connect ventilator devices, multi-parameter monitors, bedside audio video devices, clinical information systems and clinical data terminal devices from different manufacturers in each ward of the hospital through the network; the multi-mode gateway is embedded with a variety of communication protocols to realize the data communication between the ventilator devices, the multi-parameter monitors, the bedside audio video devices, the clinical information systems and the data acquisition and standardization unit, and configured to realize the data communication between the application software system and the clinical data terminal devices where the patients are located;
the master control server is embedded with a multi-task operating system and an application software system running on the multi-task operating system, is configured to control the operation of the application software system.
